(19) United States
(12) Patent Application Publication
(10) Pub. No.: US 2026/0034181 A1
(43) Pub. Date: Feb. 5, 2026

(54) ROSEBURIA INULINIVORANS FOR USE IN IMPROVING MUSCLE MASS AND STRENGTH

(71) Applicants: ACADEMISCH ZIEKENHUIS LEIDEN (H.O.D.N. LUMC), Leiden (NL); UNIVERSITY OF GRANADA, Granada (ES)

(72) Inventors: Milena SCHÖNKE, Leiden (NL); Patrick Cornelis Nicolaas RENSEN, Leiden (NL); Jonatan Ruiz RUIZ, Granada (ES); Lourdes ORTIZ ALVAREZ, Granada (ES); Borja Manuel MARTINEZ TELLEZ, Leiden (NL)

(21) Appl. No.: 18/998,818

(22) PCT Filed: Jul. 27, 2023
(86) PCT No.: PCT/NL2023/050404

§ 371 Date: Jan. 27, 2025

(30) Foreign Application Priority Data
Jul. 29, 2022 (NL) 2032642

(51) Int. Cl. A61K 35/741; A61P 21/00

(52) U.S. Cl. CPC A61K 35/741; A61P 21/00

(57) ABSTRACT

The present invention provides methods of increasing Roseburia inulinivorans in a subject for improving muscular strength, muscle mass and/or pulmonary capacity. The invention also provides Roseburia inulinivorans and compositions thereof for use in such methods. The methods may be for therapeutic treatment of muscle related disorders or reduced muscle strength. Alternatively, the methods and compositions may be for cosmetic purposes such as enhancing athletic ability in a subject.

The present invention provides methods of increasing Roseburia inulinivorans in a subject for improving muscular mass, muscle strength, and pulmonary capacity. The invention also provides Roseburia inulinivorans and compositions thereof for use in such methods. The methods may be for therapeutic treatment of muscle related disorders or reduced muscle strength. Alternatively, the methods and compositions may be for cosmetic purposes such as enhancing athletic ability in a subject.

### BACKGROUND

Low muscular strength in late adolescence is associated with a 35% higher risk of premature mortality due to any cause^{1,2} Mainly due to a lack of strength in muscles, commonly known as sarcopenia, 42% of elderly people fall each year leading to serious health consequences, and in some occasions, to death. The causes of low muscular strength are often unclear. Lack of muscle strength may be caused by lack of exercise, or by genetic disorders such as Duchenne or myotonic dystrophies, of which the prevalence is around 25.1 cases per 100,000 persons in the European population³. To date, there is no pharmacological therapy able to improve muscular strength beyond exercise training. However, for many people exercise is non-motivating, painful and/or not accessible, stressing the need for alternative strategies for improving muscular strength and/or muscle mass.

Due to the development of cutting-edge techniques, it has recently been discovered that the large intestine harbours trillions of microorganisms (i.e., gut microbiota). Thus far the biological functions of only ˜0.001% of these microorganisms have been revealed. For instance, oral supplementation of the human gut bacterium Veillonella atyipica to mice increased their running capacity⁵, and oral supplementation of Akkermansia muciniphila in patients with obesity reduced adiposity, glucose levels and blood markers of fatty liver disease⁶. These discoveries demonstrate the opportunity and feasibility of applying human gut bacteria to improve metabolic health.

U.S. Ser. No. 10/537,597B2 describes the use of Akkermansia muciniphila for treating inflammatory disorders. EP2774616B1 describes the use of Roseburia inulinivorans for treating obesity. It was found that Roseburia is capable of inhibiting body weight growth, reducing blood glucose, improving the tolerance ability of blood glucose, and ameliorating diabetes and obesity. However, there is no mention of improving muscle strength or muscle mass in subjects.

In addition, athletes require high levels of muscular strength and thus also muscle mass in order to achieve goals and compete against other athletes. The most common option to increase muscle strength is exercise training. However, in order to maximise gains in muscle mass and strength, athletes may also administer compounds such as anabolic steroids that may be detrimental to their health.

WO2017/180501 describes the use of a probiotic including one or more bacterial species of the genus Veillonella, Faecalibacterium, Phascolarctobacteria, Oscillospira, Ruminococcus, Bacteroides, Blautia, Dialister, or bacteria of the Christensenellaceae family and Cyanobacteria phylum for improving or maintaining athletic training, performance or recovery but makes no mention of improving muscular strength or muscle mass.

Furthermore, physical appearance is now more important than previously with the mass use of social media. In addition, there are some people who may be unable to exercise or sufficiently exercise to improve their physical appearance due non-medical reasons. For example, people who work long hours or have lives or lifestyles that do not allow for them to exercise.

There is a need for improved methods of improving muscle mass and/or muscular strength in subjects. There is also a need for compositions and probiotics for use in such methods.

There is a need for improved methods of altering physical appearance in subjects. There is also a need for compositions and probiotics for use in such methods.

There is a need for improved methods of improving athletic ability in subjects. There is also a need for compositions and probiotics for use in such methods.

### BRIEF SUMMARY OF THE DISCLOSURE

The invention is based on the surprising finding that the bacterial species Roseburia inulinivorans improves muscular strength, muscle mass and pulmonary capacity in subjects.

It is an aim of the invention to provide Roseburia inulinivorans, compositions which increase the population of Roseburia inulinivorans in a subject or a postbiotic composition formed by culturing Roseburia inulinivorans for use in methods of improving muscular strength in a subject.

It is an aim of the invention to provide Roseburia inulinivorans, compositions which increase the population of Roseburia inulinivorans in a subject or a postbiotic composition formed by culturing Roseburia inulinivorans for use in methods of improving muscle mass in a subject.

It is an aim of the invention to provide Roseburia inulinivorans, compositions which increase the population of Roseburia inulinivorans in a subject or a postbiotic composition formed by culturing Roseburia inulinivorans for use in methods of improving pulmonary capacity in a subject.

It is an aim of the invention to provide Roseburia inulinivorans, compositions which increase the population of Roseburia inulinivorans in a subject or a postbiotic composition formed by culturing Roseburia inulinivorans for use in methods of treating muscle-related disorders in a subject.

It is an aim of the invention to provide Roseburia inulinivorans, compositions which increase the population of Roseburia inulinivorans in a subject or a postbiotic composition formed by culturing Roseburia inulinivorans for use in methods of treating neurogenic muscular disorders in a subject.

It is an aim of the invention to provide Roseburia inulinivorans, compositions which increase the population of Roseburia inulinivorans in a subject or a postbiotic composition formed by culturing Roseburia inulinivorans for use in methods of improving physical appearance of a subject for cosmetic purposes.

It is an aim of the invention to provide Roseburia inulinivorans, compositions which increase the population of Roseburia inulinivorans in a subject or a postbiotic composition formed by culturing Roseburia inulinivorans for use in methods of improving or maintaining athletic training, performance or recovery of a subject for cosmetic purposes.

In one aspect of the invention there is provided Roseburia inulinivorans or a postbiotic composition formed by culturing Roseburia inulinivorans for use in improving muscular strength; muscle mass; and/or pulmonary capacity in a subject.

In one aspect of the invention there is provided a method of improving or maintaining athletic training, performance or recovery by a subject comprising increasing the population of Roseburia inulinivorans in the subject. In some embodiments, the method is non-therapeutic. In certain embodiments, the method comprises administering to the subject an effective amount of a composition configured to increase the population of endogenous Roseburia inulinivorans in the subject. In certain embodiments, the composition configured to increase the population of endogenous Roseburia inulinivorans in the subject comprises a prebiotic.

In one aspect of the invention there is provided a method of improving muscular strength, muscle mass, and/or pulmonary capacity in a subject comprising increasing the population of Roseburia inulinivorans in the subject. In certain embodiments, the method comprises administering to the subject an effective amount of a composition configured to increase the population of endogenous Roseburia inulinivorans in the subject. In certain embodiments, the composition configured to increase the population of endogenous Roseburia inulinivorans in the subject comprises a prebiotic.

In one aspect of the invention there is provided, use of Roseburia inulinivorans for improving in a subject, muscular strength, muscle mass; and/or, pulmonary capacity.

In one aspect of the invention there is provided, use of Roseburia inulinivorans for improving or maintaining athletic training, performance or recovery by a subject.

In one aspect of the invention there is provided, use of Roseburia inulinivorans for the manufacture of a medicament for improving in a subject, muscular strength, muscle mass, and/or, pulmonary capacity; or improving or maintaining athletic training, performance or recovery by a subject.

In one aspect there is provided a postbiotic composition as described herein for use in improving:
muscular strength;
muscle mass; and/or
pulmonary capacity
in a subject.

In certain embodiments, the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, injury or ageing or combination thereof. In certain embodiments, the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, or injury or combination thereof.

In certain embodiments, the subject suffers from atony, muscular dystrophy, myasthenia, myotonia, cachexia, sarcopenia, acardiotrophy or frailty, or any combination thereof.

In certain embodiments, the Roseburia inulinivorans comprise Roseburia inulinivorans DSM16841.

In certain embodiments, the subject suffers from a muscle-related disorder, reduced muscle mass and/or reduced muscle strength.

In certain embodiments, the subject suffers from a muscular disease and/or a neurogenic muscular disease.

In certain embodiments, the muscle-related disorder or muscular disease is selected from the group consisting of:
atony, muscular atrophy, muscular dystrophy, muscle degeneration, myasthenia, myotonia, amyotrophic lateral sclerosis, spinal muscular atrophy, cachexia, sarcopenia, acardiotrophy and frailty, or any combination thereof.

In certain embodiments, the reduced muscle mass and/or the reduced muscular strength is caused by the muscle-related disorder.

In certain embodiments, the reduced muscle mass and/or the reduced muscular strength is caused by a sequelae of immobilization, chronic disease, cancer, or injury.

In certain embodiments, improving muscle strength, muscle mass, and/or pulmonary capacity in the subject compensates for wasting resulting from the muscle-related disorder, immobilization, and/or old age.

In certain embodiments, improving muscle strength, muscle mass, and/or pulmonary capacity is for cosmetic purposes. In such embodiments, the subject does not suffer from a pathological disease or condition that affects muscle strength and/or muscle mass.

In certain embodiments, the population of Roseburia inulinivorans in the subject is increased by administration of an effective amount of Roseburia inulinivorans to the subject.

In certain embodiments, the Roseburia inulinivorans in the subject leads to an improvement of handgrip strength in the subject.

In certain embodiments, the Roseburia inulinivorans in the subject leads to an improvement of bench press strength in the subject.

In certain embodiments, the Roseburia inulinivorans in the subject leads to an improvement of leg press strength in the subject.

In certain embodiments, the Roseburia inulinivorans in the subject leads to an improvement of VO₂ max in the subject.

In certain embodiments, the Roseburia inulinivorans is administered as part of a composition.

In certain embodiments, the composition is a probiotic composition.

In certain embodiments, the composition is a postbiotic composition. In certain embodiments, the composition is a postbiotic composition and the Roseburia inulinivorans is non-viable.

In certain embodiments, the composition is a pharmaceutical composition.

In certain embodiments, the composition is comestible.

In one aspect of the invention there is provided a prebiotic composition for increasing the population of endogenous Roseburia inulinivorans in a subject for use in improving muscular strength; muscle mass; and/or pulmonary capacity in a subject.

In certain embodiments, the prebiotic composition comprises one or more of:
inulin; melibiose; glucose; sucrose; starch; acetate; lactate; butyrate; propionate; fructo-oligosaccharides; fucose; and/or methionine.

In another aspect of the invention there is provided a prebiotic composition as described herein for increasing the population of Roseburia inulinivorans in a subject in methods as described herein.

In one aspect of the invention there is provided a postbiotic composition comprising:
a culture extract of a culture of Roseburia inulinivorans.

In certain embodiments, the culture extract comprises non-viable Roseburia inulinivorans or a part thereof.

In certain embodiments, the culture extract comprises compounds extracted and/or secreted from Roseburia inulinivorans.

In certain embodiments, the Roseburia inulinivorans is as described herein.

In one aspect there is provided a method of improving or maintaining athletic training, performance or recovery by a subject comprising administering a postbiotic composition as described herein.

In one aspect there is provided a method of improving muscular strength, muscle mass, and/or pulmonary capacity in a subject comprising administering a postbiotic composition as described herein.

In certain embodiments, the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, injury or ageing or combination thereof. In certain embodiments, the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, or injury or combination thereof.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows that oral gavage with Roseburia inulinivorans-DSM16841 significantly increases forelimb grip strength after 4, 6 and 8 weeks (left), and causes a higher relative increase in forelimb grip strength after 8 weeks in mice (right). KGF: kilogram force.

FIG. 2 shows that individuals with high handgrip strength (H; ranges from 32.55 to 54.05 kg; n=30) have significantly higher relative abundance of the Roseburia genus than individuals with medium handgrip strength (M; ranges from 27.55 to 32.20 kg; n=30) or low handgrip strength (L; ranges from 19.25 to 27.50 kg; n=30).

FIG. 3 shows that the relative abundance of Roseburia inulinivorans-DSM16841 and Roseburia intestinalis-DSM 14610 positively correlates with lean body mass (LBM), lean mass index (LMI), handgrip strength, leg press strength, bench press strength and VO2max in young adults (n=90). BMI: body mass index; FBM: fat body mass; FM %: fat mass percentage; VAT: visceral adipose tissue.

FIG. 4 shows that oral gavage with Roseburia inulinivorans-DSM16841 does not affect the caecum short-chain fatty acids levels (e.g., acetate, butyrate or propionate) but significantly decreases the caecum levels of methionine compared to the other treatments. * means P<0.05. Data are expressed as Probabilistic Quotient Normalization (PQN) values.

FIG. 5 shows that oral gavage with Roseburia inulinivorans-DSM16841 (RIL) on the plasma metabolome (FIG. 5A) compared to the control group. Specifically, alterations are observed in various pathways, including protein digestion and absorption, biosynthesis of amino acids, neuroactive ligand-receptor interaction, central carbon metabolism in cancer, ABC transporters, Pantothenate and CoA biosynthesis, mineral absorption, aminoacyl-tRNA biosynthesis, beta-Alanine metabolism, and Fatty acid biosynthesis (FIG. 5B). In contrast, Roseburia intestinalis-DSM14610 (RIT; FIG. 5C) and Roseburia faecis-DSM16840 (RF; FIG. 5D) induce only minor changes in the plasma metabolome when compared to the control group.

FIG. 6 shows that oral gavage with Roseburia inulinivorans-DSM16841 (RIL) on the muscle metabolome (FIG. 6A) compared to the control group. Specifically, alterations are observed in various pathways, including purine metabolism, Neuroactive ligand-receptor interaction, platelet activation, regulation of lipolysis in adipocytes, Parkinson disease, vascular smooth muscle contraction, renin secretion, pentosephosphate, ABC transporters and Pentose and glucuronate interconversions (FIG. 6B). In contrast, Roseburia intestinalis-DSM14610 (RIT; FIG. 6C) and Roseburia faecis-DSM16840 (RF; FIG. 6D) induce only minor changes in the plasma metabolome when compared to the control group.

FIG. 7 shows that oral gavage with Roseburia inulinivorans-DSM16841 (RIL) tends to increase muscle fiber size compared to control and significantly increases compared to Roseburia intestinalis-DSM14610 (RIT) (FIG. 7A). It was also observed that Roseburia inulinivorans-DSM16841 (RIL) tends to increase the muscle fiber size larger than 5000 μm² (FIG. 7B).

FIG. 8 shows the variation in the abundance of Roseburia faecis-DSM16840, Roseburia intestinalis-DSM14610, and Roseburia inulinivorans-DSM16841 over the course of a lifespan, utilizing publicly available datasets. Correlation coefficients are denoted as “R,” and p-values less than 0.05 are considered statistically significant. The figure demonstrates a significant negative correlation between the relative abundance of Roseburia inulinivorans-DSM16841 and age during both the adult (n=3,257, FIG. 8E) and senior (n=255, FIG. 8F) life stages. This indicates a decrease in the relative abundance of Roseburia inulinivorans-DSM16841 over the lifespan. The curves exhibit locally weighted scatterplot smoothing (LOESS), while the shaded areas represent the 95% confidence intervals. The Y-axes present the relative abundance on a log 10 scale with a pseudocount of 1e-6 added to all missing samples.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5 to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

The invention is based on the surprising finding that the bacterial species Roseburia inulinivorans is capable of improving muscular strength, muscle mass and/or pulmonary capacity in subjects. Roseburia inulinivorans is a member of the family Firmicutes that contributes to butyrate formation from a variety of dietary polysaccharide substrates in the human large intestine. Roseburia inulinivorans can comprise up to 2% of the gut microbiota in human subjects. Roseburia inulinivorans is anaerobic, Gram-negative or Gram-variable, slightly curved rod-shaped with cells ranging in size from 0.5 to 5.0 micrometres. It does not form spores, is motile, has clusters of flagella at the proximal end of the concave of the bacterial cell, is capable of fermenting carbohydrates include wood sugar, galactose, raffinose, glucose, maltose, cellobiose, sucrose, starch and glycogen, and mainly produces or only produces butyric acid. Exemplar cultivation conditions for Roseburia inulinivorans may be: in M2GSC media, at 37° C., anaerobic, for 2-5 days or YCFA medium (pH6.8) 19 solidified with 1.5% bacteriological agar or in liquid YCFAC medium incubated at 37° C. overnight. It will clear to those skilled on the art that other culture conditions and media may be used.

In some examples, the Roseburia inulinivorans may have a genome sequence comprising at least 70% sequence identity to the genome identified by the GenBank sequence reference GenBank GCA_000174195.1. For example, at least 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity the genome identified by the GenBank sequence reference GenBank GCA_000174195, GenBank GCA_000174195.1 or GenBank GCA_020731525.1. In some examples, the Roseburia inulinivorans may have a genome comprising at least 80% sequence identity to the genome identified by the GenBank sequence reference GenBank GCA_000174195, GenBank GCA_000174195.1 or GenBank GCA_020731525.1 In some examples, the Roseburia inulinivorans may have a genome comprising at least 85% sequence identity to the genome identified by the GenBank sequence reference GenBank GCA_000174195, GenBank GCA_000174195.1 or GenBank GCA_020731525.1. In some examples, the Roseburia inulinivorans may have a genome comprising at least 90% sequence identity to the genome identified by the GenBank sequence reference GenBank GCA_000174195, GenBank GCA_000174195.1 or GenBank GCA_020731525.1. In some examples, the Roseburia inulinivorans may have a genome comprising at least 95% sequence identity to the genome identified by the GenBank sequence reference GenBank GCA_000174195, GenBank GCA_000174195.1 or GenBank GCA_020731525.1. In some examples, the Roseburia inulinivorans may have a genome comprising at least 99% sequence identity to the genome identified by the GenBank sequence reference GenBank GCA_000174195, GenBank GCA_000174195.1 or GenBank GCA_020731525.1.

“Percentage (%) sequence identity” with respect to a genome of bacterial species described herein refers to the percentage of nucleic acid residues in a candidate sequence that are identical with the nucleic acid residues of a reference nucleic acid sequence. Alignment for purposes of determining percentage sequence identity can be achieved in various ways, for instance, using publicly available computer software such as BLAST, BLAST-2, ALIGN, ALIGN-2 or Megalign (DNASTAR) software.

In some examples, the Roseburia inulinivorans comprises the strain DSM16841, which is available from DSMZ (Deutsche Sammlung von Mikroorganismen and Zellkulturen GmbH). In some examples, the Roseburia inulinivorans is a variant strain of DSM16841. A “variant strain” refers to a Roseburia inulinivorans strain derived from or originating from DSM16841. For example, a variant strain may include one or more conservative or non-conservative modifications such as genetic mutations, but still maintains the same characteristics and advantageous properties of the parent strain (origin strain) as described herein.

The Roseburia inulinivorans may be obtained commercially and/or produced by a fermentation process. The Roseburia inulinivorans may be grown on a suitable medium, under conditions of strict anaerobiosis, in the presence of a carbon-based substrate and/or a carbon based energy source; the bacterial cells are recovered; and the bacterial cells may be packaged. Bacterial cells may be recovered by centrifugation, for example between 2,000 g and 15,000 g. The bacterial cells may be washed in, for example, a phosphate buffer, by resuspension of the cells, agitation, and a further centrifugation step. The Roseburia inulinivorans may be stored in a storage buffer under suitable conditions, for example in phosphate buffer comprising glycerol and stored at −80° C.

In some examples, the Roseburia inulinivorans may be dried. The drying of bacterial strains after production by fermentation or other methods is known to those of skill in the art, for example as described in EP0818529 hereby incorporated by reference in its entirety wherein a drying process of pulverization is described. Additional methods are described in WO0144440, which is also incorporated by reference in its entirety. The Roseburia inulinivorans may be concentrated from a medium and dried by spray drying, fluidized bed drying, lyophilization (freeze drying) or other drying process. Roseburia inulinivorans can be mixed, for example, with a carrier material such as a carbohydrate such as sucrose, lactose or maltodextrin, a lipid or a protein, for example milk powder during or before drying.

The Roseburia inulinivorans need not necessarily be present in a dried form. It may also be suitable to mix the bacteria directly after fermentation in a composition such as a comestible product and, optionally, perform a drying process thereafter. Such an approach is disclosed in WO2002065840 which is incorporated by reference in its entirety. Likewise, the Roseburia inulinivorans as described herein may also be consumed directly after fermentation. Further processing, for example, for the sake of the manufacture of convenient comestible products, is not a precondition for the beneficial properties of the bacterial strains provided.

The Roseburia inulinivorans may be formulated as part of a composition. For example, the Roseburia inulinivorans may be part of a probiotic composition. The term “probiotic composition” refers to a composition containing at least one live Roseburia inulinivorans bacterial strain as described herein that can beneficially affect a subject by altering the subject's gut or intestinal microbial balance, composition and/or functionality without causing disease. Probiotics are intended as a supplement and may be administered regularly for effects to be maintained or persist. After administration, for example by ingestion, probiotics typically adhere to a tissue of the host, such as the wall of the intestine or gut or other tissue. Once attached, the desirable bacteria are capable of multiplying and colonizing, thereby altering the microbiota of a subject.

As used herein, the terms “gut flora”, “gastrointestinal flora”, “intestinal flora”, “gut microbiome”, “intestinal microbiome”, and “microbiome” are interchangeable and are intended to represent the normal, naturally occurring bacterial population present in the gastric and intestinal systems of healthy subjects. It is meant to reflect both the variety of bacterial species and the concentration of bacterial species found in a healthy subject.

Probiotics are sometimes combined with prebiotics (where the combination may be referred to as “symbiotic”) which include one or more non-digestible dietary supplements, which modify the balance of the intestinal microbiota, stimulating the growth and/or activity of beneficial microorganisms such as Roseburia inulinivorans as described herein and suppressing potentially deleterious microorganisms. Exemplary supplements include oligosaccharides (fructooligosaccharides, galactooligosaccharides); inulin, lactulose, lactitol and select bacterial strains that produce nutrients that promote the growth of beneficial bacteria, such as within the intestinal tract. For example, the Roseburia inulinivorans may be included in a symbiotic composition or administered in conjunction with prebiotics that promote within an individual the proliferation of the Roseburia inulinivorans as described herein. The term “prebiotic” as used herein can be a general term to refer to chemicals and or ingredients that can affect the growth and/or activity of microorganisms in a host (e.g. can allow for specific changes in the composition and/or activity in the microbiome). In certain instances, prebiotic may refer to food ingredients or bacterial producing ingredients that are not readily digestible by endogenous host enzymes and confer beneficial effects on an organism that consumes them by selectively stimulating the growth and/or activity of a limited range of beneficial microorganisms, such as endogenous Roseburia inulinivorans in a subject's gut.

In some examples, the methods and uses described herein comprise administering a prebiotic composition to a subject. The prebiotic composition may include compounds that increase the population Roseburia inulinivorans already present in the subject (i.e., endogenous Roseburia inulinivorans). Some non-limiting examples of components of prebiotic compositions can include: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, xylooligosaccharides (XOS), locust bean gum, P-glucan, and methylcellulose. Prebiotics can be found in foods (e.g. acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, banana), and breast milk. Prebiotics can also be administered in other forms (e.g. capsule or dietary supplement).

In some examples, the prebiotic may include any compound or substance that is known to increase an endogenous population of Roseburia inulinivorans in a subject. In some examples, the prebiotic may include one or more compounds that are a metabolite and/or cross-metabolite of Roseburia inulinivorans. Examples of compounds that may increase the population of Roseburia inulinivorans can be found in, for example, Duncan S H, Aminov R I, Scott K P, Louis P, Stanton T B, Flint H J. Proposal of Roseburia faecis sp. nov., Roseburia hominis sp. nov. and Roseburia inulinivorans sp. nov., based on isolates from human faeces. Int J Syst Evol Microbiol. 2006 October; 56(Pt 10):2437-2441. doi: 10.1099/ijs.0.64098-0. PMID: 17012576., Scott, Karen P et al. “Substrate-driven gene expression in Roseburia inulinivorans: importance of inducible enzymes in the utilization of inulin and starch.” Proceedings of the National Academy of Sciences of the United States of America vol. 108 Suppl 1, Suppl 1 (2011): 4672-9. doi:10.1073/pnas.1000091107, Scott, Karen P et al. “Whole-genome transcription profiling reveals genes up-regulated by growth on fucose in the human gut bacterium “Roseburia inulinivorans”.” Journal of bacteriology vol. 188, 12 (2006): 4340-9. doi:10.1128/JB.00137-06 and Louis, P. and Flint, H. J. (2017), Formation of propionate and butyrate by the human colonic microbiota. Environ Microbiol, 19: 29-41.

For example, the prebiotic may include one or more of inulin, melibiose, glucose, sucrose, starch (such as amylopection), acetate, lactate, butyrate, propionate, fructo-oligosaccharides, fucose and/or methionine. Fructo-oligosaccharides refers to oligosaccharides that are composed of linear chains of fructose units, linked by beta (2-1) bonds. They occur naturally in plants such as onion, chicory, garlic, asparagus, banana, artichoke, among many others.

For a probiotic to successfully exert its benefit on a subject's gut microbiota it should be able to remain viable during storage and also be capable of surviving, and potentially colonizing, the host's intestinal environment. Therefore, the probiotic composition should contain a concentration of live Roseburia inulinivorans that is effective in causing benefits in the subject.

In some examples, the Roseburia inulinivorans is formulated in a pharmaceutical composition. A pharmaceutical composition may include the probiotic composition in combination with a pharmaceutically acceptable carrier which may include one or more excipients.

Pharmaceutically acceptable carriers or excipients are well known in the pharmaceutical art, and are described, for example, in Remington's Pharmaceutical Sciences, Mack Publishing Co. (A. R. Gennaro edit. 1985). For example, saline and phosphate-buffered saline at physiological pH may be used. Stabilizers, dyes and even flavouring agents may be provided in the probiotic or pharmaceutical compositions described herein.

A pharmaceutical composition may comprise an effective amount of Roseburia inulinivorans, a metabolite thereof, and/or one or more pharmaceutically acceptable carriers, excipients, adjuvants and/or thinners.

Suitable carriers, excipients, and/or thinners may comprise lactose, glucose, sucrose, sorbitol, mannose, starch, gum arabic, calcium phosphate, alginate, gelatin, calcium silicate, fine crystalline cellulose, polyvinyl pyrrolidone (PVP), cellulose, water, syrup, methyl cellulose, methyl p-hydroxybenzoate, propyl para-hydroxybenzoate esters, talc, magnesium stearate or mineral oil. Pharmaceutical compositions can further comprise lubricants, wetting agents, emulsifiers, suspension stabilizers, preservatives, sweeteners, spices, etc. The pharmaceutical composition may be manufactured so that the active constituent of the pharmaceutical composition, i.e., the Roseburia inulinivorans contained therein can be protected from gastric acid and go through the stomach intact by the various methods known in the art. Examples of solid carriers include starch, lactose, dicalcium phosphate, microcrystalline cellulose, sucrose and white clay, and examples of liquid carriers includes media, polyethylene glycol, non-ionic surfactants, and edible oils (such as corn oil, peanut oil, and sesame oil). The compositions described herein can also include adjuvants such as, flavoring agents, pigments, preservatives and antioxidants such as vitamin E, vitamin C, BHT and BHA.

The Roseburia inulinivorans or a composition thereof as described herein may be comestible. For example, formed into an ingestible liquid medium, a gel medium, a food stuff, a food product, a freeze dried product or powder, a yogurt, a pill, a tablet, a capsule, a gelatin capsule, a caplet, a chewable formulation, a dissolvable formulation and the like, such as for oral administration. Compositions described herein may also contain conventional food supplement fillers and extenders such as, for example, a flour, a binder, a neutraceutical compound or formulation, a prebiotic, an amino acid, a vitamin, or a mineral. The compositions described herein may be added to a liquid product, for example, a beverage or a drink. The liquid product comprising the Roseburia inulinivorans should be consumed relatively quickly upon addition of the Roseburia inulinivorans. However, if the Roseburia inulinivorans are added to a shelf-stable product, quick consumption may not be necessary, so long as the Roseburia inulinivorans are stable in the beverage or the drink.

The compositions described herein may be dried with a food product as described in WO98/10666, which is incorporated herein by reference in its entirety. Accordingly, the Roseburia inulinivorans may be dried at the same time with juices, milk-based products or vegetable milks, for example, yielding a dried product already comprising the Roseburia inulinivorans. This product may later be reconstituted with an aqueous liquid such as milk or water.

The Roseburia inulinivorans and/or prebiotic can be administrated orally or by other suitable routes for Roseburia inulinivorans. When administered orally the Roseburia inulinivorans, compositions thereof or prebiotics may be in the form of capsules, tablets, powders, granules, solutions, or suspensions. The Roseburia inulinivorans can be mixed with conventional excipients, such as gelatin, starch, lactose, magnesium stearate, talc, gum arabic and the like. It may also be advantageous to use less conventional excipients that, for example, make it possible to increase the ability of Roseburia inulinivorans to be active in the gut. For example, cellobiose, maltose, mannose, salicine, trehalose, amygdalin, arabinose, melobiose, rhamnose and/or xylose may be added. This list is not exhaustive and the substrates may be chosen or changed depending on circumstances. Additionally, a capsule, pill, tablet, or syrup for oral administration should be stored in a manner so as to preserve its efficacy. Methods of storage for probiotic compositions include but are not limited to refrigeration, freezing, and storing at room temperature. If stored at room temperature, the Roseburia inulinivorans or compositions thereof should be stored in an air tight container.

Roseburia inulinivorans should arrive intactly in the small and large intestine the latter of which may be colonized. The Roseburia inulinivorans may be microencapsulated or enterically coated to provide a release profile that targets replacement or alteration of live Roseburia inulinivorans at a pre-determined location within the gastrointestinal tract of a subject. Such microencapsulation formulations and techniques may protect the live Roseburia inulinivorans from the digestive actions of the stomach, duodenum, and jejunum of the intestine and allow administration, delivery or release to the gut or ileum of a subject. Microencapsulated live Roseburia inulinivorans and compositions thereof may be in various dosage forms, and they can be co-administered with drugs, foods, nutrients, vitamins, other beneficial substances, prebiotics, and other therapeutic agents such as pH encapsulated glucose, lipids or proteins that release in the distal small intestine at pH values between 7.0 and 8.0 in an amount sufficient to achieve the desired effect in a subject. For example, at least two coatings may be used to cover a tablet or capsule like form comprising the Roseburia inulinivorans, wherein the outside coating is degraded in a pH environment of 5 to 6 and the inside coating is degraded in a pH environment of about 7 thereby releasing the Roseburia inulinivorans in the ileum area. An exemplary coating may include one or more of poly(dl-lactide-co-glycolide) chitosan, casein, chitosan (Chi) stabilized with PVA (poly-vinylic alcohol), a lipid, an alginate, carboxymethylethylcellulose (CMEC), cellulose acetate trimellitiate (CAT), hydroxypropylmethyl cellulose phthalate (HPMCP), hydroxypropylmethyl cellulose, ethyl cellulose, color con, food glaze and mixtures of hydroxypropylmethyl cellulose and ethyl cellulose, polyvinyl acetate phthalate (PVAP), cellulose acetate phthalate (CAP), shellac, copolymers of methacrylic acid and ethyl acrylate, and copolymers of methacrylic acid and ethyl acrylate to which a monomer of methylacrylate has been added during polymerization, and other methacrylic resins that are commercially available under the tradename Eudragit® (Rohm Pharma; Westerstadt, Germany), including Eudragit® L30D-55 and L100-55 (soluble at pH 5.5 and above), Eudragit® L-100 (soluble at pH 6.0 and above), Eudragit® S (soluble at pH 7.0 and above, as a result of a higher degree of esterification), and Eudragits® NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, amylose and guar gum; and zein. Combinations of different coating materials may also be used. Multi-layer coatings using different polymers may also be applied. The preferred coating weights for particular coating materials may be readily determined by those skilled in the art by evaluating individual release profiles for tablets, beads and granules prepared with different quantities of various coating materials. It is the combination of materials, method and form of application that produce the desired release.

The compositions described herein may be administered once or more per day to a subject. The administration amount can vary depending on the body weight of the subject, the severity of a subject's condition, the subject's desired outcomes (for example when used for cosmetic purposes), supplemental active ingredients included and microorganisms used therein. In addition, it is possible to divide up a daily administration amount and to administer continuously, if needed. Suitable dosages of Roseburia inulinivorans or compositions thereof may be provided orally at a dosage rate of at least 10 milligrams to at least 1000 milligrams per day. The dosage rate, effective as a food supplement and for establishing or increasing Roseburia inulinivorans in the intestinal tract of a subject may be determined by known methods in the art.

In some examples, Roseburia inulinivorans or compositions thereof may be administered 1, 2 3, 4, 5 or more times per week for a specified time period. For example, the specified time period may be at least one week. In some examples, the specified time period may be at least 1, at least 2, at least 3, at least 4, at least 5, at least 6, at least 7, at least 8 weeks or more. In some examples, administration may be continuous, for example a subject may continually take doses of Roseburia inulinivorans or compositions thereof even after effects the beneficial effects have been achieved. In the context of medical conditions and diseases that may reduce muscle mass and/or strength, continuous administration may help prevent or reduce the muscle wasting effects of the condition or disease.

An “effective amount” refers to an amount of a Roseburia inulinivorans sufficient to achieve the desired function or activity in the subject and is acceptable to the subject (i.e. does not cause adverse effects in the subject). For example, an effective amount may be an amount of Roseburia inulinivorans that provides some improvement or benefit to a subject having a disease, condition, or disorder in which a loss of muscle mass and/or muscle strength occurs. In relation to cosmetic uses of Roseburia inulinivorans as described herein an effective amount may be an amount that leads to a subject achieving a set goal (i.e. a specific gain in muscle mass, muscle strength or change in appearance in a defined time period). Thus, an “effective amount” or “therapeutically effective amount” with reference to therapeutic uses and methods is an amount that provides some alleviation, mitigation, and/or decrease in at least one clinical symptom of the disease, condition, or disorder in which a loss of muscle mass and/or muscle strength occurs. Those skilled in the art will appreciate that therapeutic effects need not be complete or curative, as long as some benefit is provided to the subject.

An effective amount may refer to an amount of Roseburia inulinivorans that is capable of at least one of increasing muscle mass (for cosmetic or therapeutic purposes); increasing muscle strength (for cosmetic or therapeutic purposes); increasing pulmonary capacity (for cosmetic or therapeutic purposes); reducing loss of muscle mass caused by a disease or condition; reducing loss of muscle strength caused by a disease or condition; preventing the loss of muscle mass (for cosmetic purposes); preventing the loss of muscle mass caused by a disease or condition; preventing the loss of muscle strength (for cosmetic purposes) and/or preventing the loss of muscle strength caused by a disease or condition.

The effective amount of Roseburia inulinivorans and/or the metabolite thereof in the preparation of the compositions described herein can vary depending on the mode of administration and the requirements of the subject. For example, the severity of a subject's condition or the subject's desired result. For example, an effective amount of Roseburia inulinivorans may be at least 10⁶ CFU/mL. For example, at least 10⁶ CFU/mL, 10⁷ CFU/mL, 10⁸ CFU/mL, 10⁹ CFU/mL, 10¹⁰ CFU/mL or more.

In some examples, Roseburia inulinivorans may be administered to a subject by way of a faecal transplant. The term “faecal transplant” as used herein refers to faecal microbiota isolated from a healthy individual that does not have a muscle-related disorder and/or reduced muscle strength, which is transplanted into a recipient subject. In some examples, the faecal transplant is processed faecal material (faecal filtrate) having reduced volume and/or faecal aroma relative to unprocessed faecal material. In some examples, the faecal transplant is a faecal bacterial sample. The term faecal transplant may also be used to refer to the process of transplantation of faecal bacteria isolated from a healthy individual into a recipient subject. The process may be also referred to as faecal microbiota transplantation (FMT), stool transplant or bacteriotherapy.

In some examples, the methods described herein comprise administering a prebiotic that increases the amount of endogenous Roseburia inulinivorans in a subject. In some examples, the methods described herein comprise administering an effective amount of Roseburia inulinivorans to a subject (e.g., administering exogenous Roseburia inulinivorans). Exogenous Roseburia inulinivorans may be obtained or produced as described herein from any source and in some case may be obtained from the subject to which it is to be administered to and produced or cultured outside of the subject prior to being administered.

### Postbiotic

In some examples, the invention provides a postbiotic composition. The International Scientific Association for Probiotics and Prebiotics defines a postbiotic as a “preparation of inanimate microorganisms and/or their components that confers a health benefit on the host” (Nature Reviews Gastroenterology & Hepatology, volume 18, pages 649-667 (2021)). The term postbiotic encompasses both metabolites of microorganisms and inanimate (i.e., inactive or non-viable) microorganisms themselves, such as non-viable bacteria. For example, the postbiotic may comprise non-viable bacteria and agents produced by the bacteria (e.g., Roseburia inulinivorans) or byproducts thereof, including compounds secreted by live bacteria or released after bacterial lysis, such as metabolites, enzymes, peptides, teichoic acids, peptidoglycan-derived muropeptides, polysaccharides, lipids, cell surface proteins, cell wall fragments or organic acids.

In some examples, the postbiotic composition does not include whole bacteria. For example, the culture extract may only include parts of Roseburia inulinivorans (i.e., fragments thereof) which have been broken down by for example by cell lysis. In some examples, the postbiotic composition only includes agents secreted by the bacteria (e.g., Roseburia inulinivorans), lysed from the bacteria or byproducts thereof. In some examples, the postbiotic composition only includes agents secreted by the bacteria (e.g., Roseburia inulinivorans). For example, the culture extract does not include intact Roseburia inulinivorans or any parts thereof.

The postbiotic compositions may be made using a culture extract. Culture extract is used to refer to the products produced by culturing bacteria and may be in the same culture media as used for culturing the bacteria, or may refer to the agents and non-viable bacteria or fragments thereof isolated from the culture media. “Fragment” may refer to cellular components, metabolites, secreted molecules and compounds resulting from the metabolism of pasteurized Roseburia inulinivorans and the like.

Fragments may be obtained, for example, by recovering the supernatant (e.g. cell extract) of a culture of Roseburia inulinivorans after pasteurization or by extracting cell components or cell fractions, metabolites or secreted compounds from a culture of Roseburia inulinivorans after pasteurization. The term fragment may also refer to a degradation product. A fragment may correspond to a component in the isolated form or to any mixture of one or more components derived from pasteurized Roseburia inulinivorans. In one example, a fragment may correspond to one or more of such components present in pasteurized Roseburia inulinivorans that is produced in another way, such as using recombinant DNA technology, in a microbial host or in any other (bio)synthetic process.

The postbiotic composition and cell extracts of the invention may include exopolysaccharides. During their growth, microorganisms produce biopolymers with different chemical properties. These biopolymers can be released outside the bacterial cell wall, forming a heterogeneous group of substances called exopolysaccharides (EPSs).

The postbiotic composition and cell extract may include enzymes such as antioxidant enzymes. For example, antioxidant enzymes, such as glutathione peroxidase (GPx), peroxide dismutase (SOD), catalase, and NADH-oxidase.

The postbiotic composition and cell extract may include cell wall fragments.

The postbiotic composition and cell extract may include short-chain fatty acids. For example, acetic, propionic, and butyric acids.

Bacteria may be made non-viable by any known means, such as by pasteurization which may leave the bacteria whole or by lysis of bacteria after culturing. As used herein, “non-viable cells” means cells that are not able to proliferate. Measurement of cell viability and proliferation may be any method known to one skilled in the art. For example, cell viability and proliferation may be assessed by spreading a solution containing pasteurized Roseburia inulinivorans across a petri dish or using any other culture methods and counting the number of clones or optical density after a determined time of incubation in optimal growth conditions. Moreover, it is also possible to determine the number of cells, including viable as well as non-viable cells at least as the integrity of the cells is not compromised, by microscopic observation. While phase-contrast microscopy is a well-known method to do so, the microbial cells can be further visualized by specific staining with dyes, fluorescent probes or antibodies. This allows facilitation of microscopic observations while the number of stained cells can be also be counted by flow cytometry.

For example, lysis may be performed by methods such as enzymatic extraction, solvent extraction, sonication, and heat. Isolation of products produced by culturing Roseburia inulinivorans may be separated from intact Roseburia inulinivorans and/or parts thereof by any suitable methods such as dialysis methods, centrifugation methods and chromatography. Other methods of producing a cell extract and postbiotic compositions are described in US20220096571A1 which is incorporated herein by reference.

Also provided herein are methods of producing a postbiotic composition as described herein. The methods may include culturing Roseburia inulinivorans in a culture media and inactivating the Roseburia inulinivorans. In some examples, inactivating includes pasteurizing the Roseburia inulinivorans. In some examples, inactivating includes in addition or alternatively, lysis of the Roseburia inulinivorans.

In some examples, the method of producing a postbiotic further includes separating intact but non-viable Roseburia inulinivorans. For example, separating may include centrifugation, dialysis and/or chromatography. In some examples, the method of producing a postbiotic further includes extracting products produced by culturing the Roseburia inulinivorans (e.g., metabolites, enzymes, peptides, teichoic acids, peptidoglycan-derived muropeptides, polysaccharides, lipids, cell surface proteins, or organic acids). Extracting refers to a process to separate products produced by culturing the Roseburia inulinivorans from intact cells and/or parts thereof after lysis.

In some examples, the postbiotic composition is a cell free supernatant. Cell-free supernatants refer to a composition that includes biologically active metabolites secreted by Roseburia inulinivorans into the surrounding liquid obtained directly from the cell culture. After an incubation period, the Roseburia inulinivorans are centrifuged and removed. The resulting mixture (supernatant) may then be filtered and/or sterilized.

In some examples, the postbiotic composition may be a pharmaceutical composition as described herein.

In some examples, the postbiotic composition may be a comestible composition.

The postbiotic compositions described herein may be used in the methods or for use in the methods described herein. For example, a method of improving or maintaining athletic training, performance or recovery by a subject comprising administering a postbiotic composition as described herein. For example, a method of improving muscular strength, muscle mass, and/or pulmonary capacity in a subject comprising administering a postbiotic composition as described herein.

In some examples, the postbiotic composition may comprise additional components such as butyrate, lactate, propionate, acetate, valerate, leucine, isoleucine, methionine, aspartate, valine, alanine, and/or threonine.

### Kits

Also provided is a kit including Roseburia inulinivorans, which may be in a composition or encapsulated form, powder, pill, tablet or capsule and the like and may be provided within a container, and optionally with a device for measuring units of the composition and/or with instructions for administration of the composition or formulation to a subject.

### Effects

Increasing the population of Roseburia inulinivorans in a subject and/or a postbiotic formed from culturing Roseburia inulinivorans may have a number of advantageous effects. The population of Roseburia inulinivorans in a subject may be increased by administering a composition to a subject that increases the endogenous Roseburia inulinivorans in a subject as described above. Alternatively or in addition, the population of Roseburia inulinivorans may be increased by administering Roseburia inulinivorans to a subject as described above.

In some examples, increasing the population of Roseburia inulinivorans in a subject and/or a postbiotic formed from culturing Roseburia inulinivorans may improve muscular strength of the subject. For example, increasing muscular strength of the subject. Muscle strength refers to the amount of force a muscle, or muscle groups in sum, can exert. Muscle strength may be evaluated by a variety of methods such as grip strength, one repetition maximum strength test, time-dependent tests of muscle endurance, time-dependent tests of muscle fatigue, time-dependent tests of muscle endurance and fatigue, walking distance, walking speed, activity of daily living and so forth.

In some examples, increasing the population of Roseburia inulinivorans in a subject and/or a postbiotic formed from culturing Roseburia inulinivorans may improve muscle mass of the subject. “Muscle mass” refers to the amount or size of muscle or muscle groups, as expressed by muscle weight, mass, area, or volume. Muscle mass may also be expressed as total lean body mass, lean body mass of a body compartment such as the leg, or cross-sectional area of a leg or arm compartment. The volume or mass of the muscle can be determined using any known or otherwise effective technique that provides muscle area, volume or mass, such as dual energy X-ray absorptiometry (DEXA), or using visual or imaging techniques such as magnetic resonance imaging (MRI) or computed tomography (CT) scans.

As used herein the term “improvement” may be used to refer to an increase. For example, an increase in muscle mass and/or muscular strength. For example, an increase may be considered a significant increase relative to that of a reference, as determined under comparable conditions. In some examples, the increase in muscular strength and/or muscle mass is at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% higher relative to a reference, as determined under comparable conditions. In some examples, the reference may be the muscular strength and/or muscle mass prior to increasing the population of Roseburia inulinivorans in the subject and/or prior to administering a postbiotic formed from culturing Roseburia inulinivorans. In some examples, wherein the Roseburia inulinivorans is used for therapeutic purposes, the reference may be an individual who suffers from the disease or condition. Improving and increasing muscle strength may include strengthening body performance, strengthening maximum endurance, increasing muscle mass, strengthening muscle recovery, reducing muscle fatigue, improving energy balance, or a combination thereof.

In some examples, such as when the subject suffers muscle wasting (e.g., wasting resulting from a muscle-related disorder, sequelae of immobilization, sequelae of chronic disease, sequelae of cancer, or sequelae of injury and/or old age) improvement may be used to refer to a decrease in the amount or rate (i.e. the amount of muscular strength and/or muscle mass lost over a time period) of muscle wasting. For example, a reduction or prevention of the amount of loss of muscle mass and/or muscular strength or a reduction of the rate of loss of muscular strength and/or muscle mass. For example, the decreased or prevented loss of muscular strength and/or decreased or prevented loss of muscle mass is at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% lower relative to a reference, as determined under comparable conditions. In some examples, the reference may be the muscular strength and/or muscle mass or the rate of loss of muscular strength and/or muscle mass prior to increasing the population of Roseburia inulinivorans in the subject and/or prior to administering a postbiotic formed from culturing Roseburia inulinivorans. In some examples, wherein the Roseburia inulinivorans is used for therapeutic purposes, the reference may be an individual who suffers from the disease or condition.

Multiple endpoints can be used to demonstrate changes in muscle mass and muscular strength. Such endpoints include the Short Physical Performance Battery, Leg Press, a directed quality of life survey, activities of daily living (ADLs), functional independence measure (FIM), functional tests and scales (e.g. walk test, stair climb, cycle ergometer), strength tests and scales (e.g. hand grip test, manual muscle testing scales), bioimpedance analysis, electromyogram, dynamometer, dual-energy X-ray absorptiometry, computed tomography tests, magnetic resonance imaging, ultrasound, muscle biopsy, muscle histology, blood/biochemistry tests, anthropometry, skin thickness measurements, body mass index assessment, and weight monitoring. Muscle strength can be assessed using bilateral limb muscles, neck muscles or abdominal muscles.

Short Physical Performance Battery (SPPB) is a multi-component measure of lower extremity function that is assessed by measures of standing balance, walking speed, and ability to rise from a chair, rated on a scale of 0-4. Walk test is an assessment of lower extremity function that times how long it takes a patient to walk a certain distance. Leg Press measures leg strength using weights and assessment of force. Multiple scales and systems are used to qualitatively assess a patient's quality of life. Dual-energy X-ray absorptiometry (DEXA) is a measure of estimated skeletal muscle mass.

A number of assays in animals can also be used to demonstrate changes in muscle mass and muscle strength. For example, the grip strength test measures an animal's strength when pulled against a grip strength meter. The inclined plane test measures an animal's ability to suspend itself. The swim test measures functional ability through a representative activity, for example swimming, and is similar to the walk test in humans. The Hindlimb Exertion Force Test (HEFT) measures the maximum force exerted following applied tail stimulus. Other physical performance tests in animals include walking speed and wheel running. These tests/models can be used alone or in any combination.

In some examples, increasing the population of Roseburia inulinivorans and/or a postbiotic formed from culturing Roseburia inulinivorans may improve handgrip strength. Handgrip strength can be quantified by measuring the amount of static force that the hand can squeeze around a dynamometer.

In some examples, increasing the population of Roseburia inulinivorans and/or a postbiotic formed from culturing Roseburia inulinivorans may improve bench press strength. Bench press strength can be quantified by a 1 repetition max test (carrying out a single bench press repetition and progressively increasing weight for after each repetition until the maximum weight that can be lifted for one repetition is known), a bench press maximum power test (subjects bench press 50% of their body weight as quickly as possible for three reps, with maximum power measured), a relative bench press test (an upper body muscular strength endurance test, using a resistance set to a certain percentage of body weight and determining the maximum number of reps that can be done at this weight), a set weight maximum bench press test (the subject performs as many bench presses as they can at a particular weight), or a bench press beep test (subjects perform as many repetitions as possible at a set weight and set cadence).

In some examples, increasing the population of Roseburia inulinivorans and/or a postbiotic formed from culturing Roseburia inulinivorans may improve leg press strength. Leg press strength can be quantified by a 1 repetition max test, a leg press maximum power test, a relative leg press test, a set weight maximum leg press test, or a leg press beep test.

In some examples, increasing the population of Roseburia inulinivorans and/or a postbiotic formed from culturing Roseburia inulinivorans may improve pulmonary capacity (also referred to as respiratory capacity or lung capacity) of a subject. The standard lung capacities are inspiratory capacity (IC), functional residual capacity (FRC), vital capacity (VC) and total lung capacity (TLC). In some examples, increasing the population of Roseburia inulinivorans may improve one or more of inspiratory capacity (IC), functional residual capacity (FRC), vital capacity (VC) and total lung capacity (TLC).

In some examples, increasing the population of Roseburia inulinivorans and/or a postbiotic formed from culturing Roseburia inulinivorans may improve VO₂ max. VO₂ max refers to the maximum amount of oxygen that an individual can utilize during intense or maximal exercise. The measurement of VO₂ max in a laboratory provides a quantitative value of endurance fitness for comparison of individual training effects and between people in endurance training. Maximal oxygen consumption reflects cardiorespiratory fitness and endurance capacity in exercise performance. Elite athletes, such as competitive distance runners, racing cyclists or Olympic cross-country skiers, can achieve VO₂ max values exceeding 90 mL/(kg·min).

Methods for measuring VO₂ max are known in the art. For example, VO₂ max may be determined by a graded exercise test while the subject is wearing a mask that allows direct measurement of the O₂ consumed and CO₂ produced. Briefly, the velocity or resistance on a treadmill, cycle ergometer or rowing ergometer in increased at regular interval (e.g. 1 min, 2 min, 3 min, 4 min, 5 min, etc.) until exhaustion (i.e. maximal effort). The amount of the increase is statistically significant, and may be at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% in comparison to a reference, as determined under comparable conditions. In some examples, the reference may be the VO₂ max prior to increasing the population of Roseburia inulinivorans in the subject and/or prior to administering a postbiotic formed from culturing Roseburia inulinivorans. In some examples, wherein the Roseburia inulinivorans is used for therapeutic purposes, the reference may be an individual who suffers from the disease or condition.

In some examples, increasing the population of Roseburia inulinivorans and/or a postbiotic formed from culturing Roseburia inulinivorans may alter amino acid levels in the caecum of a subject. For example, increase levels of alanine, increase levels of threonine, decrease levels of valine and/or decrease levels of methionine.

In some examples, increasing the population of Roseburia inulinivorans and/or a postbiotic formed from culturing Roseburia inulinivorans may alter the proteome of a subject. For example, increasing the population of Roseburia inulinivorans and/or a postbiotic formed from culturing Roseburia inulinivorans may alter expression of genes involved in protein digestion and absorption. For example, increasing the population of Roseburia inulinivorans and/or a postbiotic formed from culturing Roseburia inulinivorans may alter expression of genes involved in one or more pathways selected from protein digestion and absorption, biosynthesis of amino acids, neuroactive ligand-receptor interaction, central carbon metabolism in cancer, AC transporters, pantothenate and CoA biosynthesis, mineral absorption, aminoacyl-tRNA metabolism, beta-alanine metabolism, and/or fatty acid biosynthesis.

For example, increasing the population of Roseburia inulinivorans and/or a postbiotic formed from culturing Roseburia inulinivorans may alter expression of genes involved in protein digestion and absorption in a subject.

In some examples, increasing the population of Roseburia inulinivorans and/or a postbiotic formed from culturing Roseburia inulinivorans may alter expression of genes involved purine metabolism in a subject.

In some examples, increasing the population of Roseburia inulinivorans and/or a postbiotic formed from culturing Roseburia inulinivorans may lead to increased muscular fibre size. For example, increasing the population of Roseburia inulinivorans and/or a postbiotic formed from culturing Roseburia inulinivorans may lead to an increase of the number of muscle fibres with a fibre size (in μm²) of at least 5000 μm². For example, an increase of at least 1%, 2%, 3%, 4%, 5%, 6%, 7%, 8%, 9%, 10%, 20%, 30%, 40%, 50%, 60%, 70%, 80%, 90%, or 100%.

### Medical Uses

Given the advantageous effects of increasing the population of Roseburia inulinivorans in a subject as described and exemplified herein, there is provided Roseburia inulinivorans for use improving muscular strength, muscle mass and/or pulmonary capacity in a subject in need thereof. For example, a subject suffering from one or more conditions or diseases. Also provided are methods of increasing the population of Roseburia inulinivorans in a subject in need thereof in order to treat diseases and conditions as described herein.

In some examples, the subject in need thereof suffers from reduced muscle strength or muscle weakness. Reduced muscle strength refers to a state in which the strength of one or more muscles is reduced. The reduced muscle strength may be limited to one muscle, one side of the body, upper or lower extremity, or may appear throughout the body. Reduced muscle strength can be determined by physical examination as well as the tests described herein. Subjective muscle weakness symptoms, including muscle fatigue and myalgia, can be quantified in an objective way through medical examinations. In some examples, a subject in need thereof suffers from muscle wasting or muscle loss. For example, reduced muscle mass.

Reduced muscle strength and/or muscle loss may be due at least in part to age (also referred to herein as an elderly subject), inactivity, injury, disease, or combinations thereof. For example, muscle loss in a subject in need thereof may be at least partially attributable to increased muscle protein degradation, decreased muscle protein synthesis, decreased muscle regeneration, or combinations thereof. In some examples, a subject in need thereof may be an elderly human, optionally a diseased elderly human. In some examples, the subject in need thereof, is a human that is undergoing a temporary or permanent period of inactivity or immobilization due to disability, disease, injury or healing from an operation. In some examples, the subject in need thereof is a human undergoing rehabilitation (i.e., physical rehabilitation) due to disease, injury, surgery, hospital admission, or combinations thereof.

In some examples, the reduced muscle strength and/or muscle loss may be a sequelae immobilization, disease, injury, surgery, hospital admission, and combinations thereof. Immobilization may be caused by any one or more of the conditions described herein. For example, a disability, a disease, an injury, surgery, hospital admission, or combinations thereof. Immobilization also includes conditions that may prohibit a subject from being able to exercise and as such lead to reduced muscle strength and/or muscle loss. For example, subjects that have temporary or permanent paralysis of at least parts of the body (e.g. paraplegics or quadriplegics), may have one or more disfigured or amputated limbs, may be sedated or may be in a coma.

Typical symptoms of decreases in muscle mass and muscle strength, include any one or any combination of general weakness, fatigue, reduction in physical activities, vulnerability to falls, functional disability, loss of autonomy, depression due to decreasing mobility, loss of appetite, malnutrition, and abnormal weight loss.

In some examples, the subject suffers from a muscular condition. For example, the subject suffers from atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, injury or ageing or combination thereof. In some examples, the subject suffers from a muscular condition such as atony, a muscular dystrophy, myasthenia, myotonia, cachexia, sarcopenia, acardiotrophy, and/or frailty.

In some examples, the subject does not suffer from a neurodegenerative condition.

In some examples, the subject suffers from a muscle-related disorder. “Muscle-related disorder” refers to any disease or condition that may cause or be caused by the reduced muscle strength and/or muscle loss. For example, muscle-related disorders may include muscular diseases (myogenic) and neurogenic muscular disorders (neurogenic).

Muscle related disorders described herein refer to conditions and diseases accompanied by a decrease in muscle strength, skeletal muscle mass, and the like. Most of the symptoms of muscle related disorders are weakness caused by atrophy and loss of muscles, especially skeletal muscles. There are two general causes of muscle atrophy or loss: one is a problem with the muscle itself (generally called myogenic disease), and the other is a problem with the nerves that move the muscle (generally referred to as neurogenic muscular disease). Myogenic diseases include muscular dystrophies (e.g., Duchenne muscular dystrophy, Becker muscular dystrophy, limb girdle muscular dystrophy, distal myopathy, congenital muscular dystrophy, myotonic dystrophy). Examples of neurogenic muscular diseases include infant spinal muscular atrophy (Welndrich-Hoffmann disease), Kugelberg-Wehlander disease, Dentatorubral-pallidoluysian atrophy (DRPLA) and the like.

Examples muscle-related disorders include atony, muscular atrophy, muscular dystrophy, muscle degeneration, myasthenia, myotonia, amyotrophic lateral sclerosis, spinal muscular atrophy, cachexia, sarcopenia, acardiotrophy and frailty, or any combination thereof.

Other diseases and disorders in which loss of muscle mass and strength has been observed include, multi-infarct dementia, stroke, trauma, infections, meningitis, encephalitis, Pick's Disease, frontal lobe degeneration, corticobasal degeneration, multiple system atrophy, progressive supranuclear palsy, Creutzfeldt-Jakob disease, Lewy body disease, neuroinflammatory disease, spinal muscular atrophy, Parkinson's Disease, Alzheimer's Disease, AIDS, Chron's Disease, Huntington's Disease, gliomas, cancers (including brain metastasis), HIV infection, HIV-1 associated dementia (HAD), HIV associated neurocognitive disorders (HAND), paralysis, multiple sclerosis (MS), CNS-associated cardiovascular disease, prion disease, metabolic disorders, surgery, burns, injury to muscle bone or nerve, obesity, diabetes (including type II diabetes mellitus), arthritis, chronic renal failure (CRF), end stage renal disease (ESRD), congestive heart failure (CHF), chronic obstructive pulmonary disease (COPD), elective joint repair, motor neuron neuropathy, osteoporosis, osteoarthritis, fatty acid liver disease, liver cirrhosis, Addison's disease, Cushing's syndrome, acute respiratory distress syndrome, steroid induced muscle wasting, myositis and scoliosis. Loss of muscle mass has also been observed in lysosomal storage diseases such as, Gaucher's disease, Pompe disease, Niemann-Pick, Hunter syndrome (MPS II), Mucopolysaccharidosis I (MPS I), GM2-gangliosidoses, Gaucher disease, Sanfilippo syndrome (MPS IIIA), Tay-Sachs disease, Sandhoff's disease, Krabbe's disease, metachromatic leukodystrophy, and Fabry disease.

In some examples, the Roseburia inulinivorans or compositions thereof are for use in treating or preventing or for use in methods of treating or preventing muscle loss and/or reduced muscular strength in subjects suffering from or at risk of a muscle related disorder as described herein. In some examples, there is provided methods of increasing the population of Roseburia inulinivorans in a subject suffering from or at risk of muscle loss and/or reduced muscular strength due to a muscle related disorder as described herein.

Atony refers to the lack of normal tone or strength of muscles. Muscle tone is defined as the continuous and passive-partial contraction of the muscle or the muscle's resistance to passive stretch during the resting state. Atony often effects organs such as the uterus (be caused by giving birth) and stomach. Atony may also be caused by anorexia nervosa, thrombosis of organ vessels, endocrine pathologies and mechanical damage.

In some examples, the Roseburia inulinivorans or compositions thereof are for use in treating or preventing or for use in methods of treating or preventing muscle loss and/or reduced muscular strength in subjects suffering from or at risk of atony. In some examples, there is provided methods of increasing the population of Roseburia inulinivorans in a subject suffering from or at risk of muscle loss and/or reduced muscular strength due to atony.

Muscular atrophy refers to the wasting (thinning) or loss of muscle tissue (i.e. loss or reduced muscle mass). Muscular atrophy may have a number of causes such as, lack of physical activity for an extended period of time, aging, alcohol-associated myopathy, burns, injuries, malnutrition, spinal cord or peripheral nerve injuries, stroke, or long-term corticosteroid therapy. Muscular atrophy may also be caused by conditions such as amyotrophic lateral sclerosis, dermatomyositis, Guillain-Barre syndrome, multiple sclerosis, muscular dystrophy, neuropathy, osteoarthritis, polio, polymyositis, rheumatoid arthritis, and spinal muscular atrophy. Muscular atrophy may be diagnosed by methods known in the art such as blood tests, X-rays, MRI, CT scan, nerve conduction studies, muscle or nerve biopsy and electromyography.

In some examples, the Roseburia inulinivorans or compositions thereof are for use in treating or preventing or for use in methods of treating or preventing muscle loss and/or reduced muscular strength in subjects suffering from or at risk of muscular atrophy. In some examples, there is provided methods of increasing the population of Roseburia inulinivorans in a subject suffering from or at risk of muscle loss and/or reduced muscular strength due to muscular atrophy.

Muscular dystrophy is a disease in which gradual muscle atrophy and muscle weakness develop, and refers to degenerative myopathy characterized by necrosis of muscle fibres pathologically. Muscle cell membrane damage leads to muscle fibre necrosis and degeneration, which cause muscle weakness and atrophy. Muscular dystrophy can be subdivided according to the extent and distribution of muscle weakness, the age at onset, the rate of progression, the severity of symptoms, and family history. Non-limiting examples of the muscular dystrophy include Duchenne muscular dystrophy, Becker muscular dystrophy, limb-girdle muscular dystrophy, Emery-Dreifuss muscular dystrophy, facioscapulohumeral muscular dystrophy, myotonic dystrophy, myotonic muscular dystrophy, oculopharyngeal muscular dystrophy, distal muscular dystrophy, and congenital muscular dystrophy.

Duchenne muscular dystrophy is generally diagnosed by muscle biopsy by genetic diagnosis (MLPA method) and/or immunohistochemical staining using an anti-dystrophin antibody. In patients with Duchenne muscular dystrophy, steroid therapy is commonly administered.

Similarly, other congenital muscular dystrophies are generally identified with genetic diagnosis (for example, Southern blotting or PCR detection of transposon insertion into the fuctin gene (causative gene) in Fukuyama type congenital muscular dystrophy) and antibodies of each causative gene. They may also be diagnosed by tissue biopsy by tissue staining.

Other muscular dystrophies, such as muscular dystrophy with slow progression of weakness, such as Becker muscular dystrophy and limb muscular dystrophy, are generally diagnosed by genetic diagnosis (for example, MLPA method in Becker type) and/or muscle biopsy by immunohistochemical staining is done.

In some examples, the Roseburia inulinivorans or compositions thereof are for use in treating or preventing or for use in methods of treating or preventing muscle loss and/or reduced muscular strength in subjects suffering from or at risk of muscular dystrophy. In some examples, there is provided methods of increasing the population of Roseburia inulinivorans in a subject suffering from or at risk of muscle loss and/or reduced muscular strength due to muscular dystrophy.

Myasthenia refers to a neuromuscular disease, also called myasthenia gravis, which is characterized by frequent occurrence of weakness and fatigue. Weakness is more noticeable during exercise and less noticeable at rest. This is caused by the circulation of self-antigens that block nicotinic acetylcholine receptors. These antibodies prevent the transmission of signals from motor neurons to muscle. There are other forms of neuromuscular disease associated with myasthenia gravis, such as Lambert-Eaton myasthenia gravis syndrome.

In some examples, the Roseburia inulinivorans or compositions thereof are for use in treating or preventing or for use in methods of treating or preventing muscle loss and/or reduced muscular strength in subjects suffering from or at risk of myasthenia. In some examples, there is provided methods of increasing the population of Roseburia inulinivorans in a subject suffering from or at risk of muscle loss and/or reduced muscular strength due to myasthenia.

Myotonia refers to any disorder or condition characterized by tonic spasm or temporary rigidity of a muscle and in particular the decreased relaxation of a muscle following a sustained contraction. Examples of disorders that exhibit myotonia include myotonic dystrophy, myotonia congenital, and paramyotonia congenital disorders.

In some examples, the Roseburia inulinivorans or compositions thereof are for use in treating or preventing or for use in methods of treating or preventing muscle loss and/or reduced muscular strength in subjects suffering from or at risk of myotonia. In some examples, there is provided methods of increasing the population of Roseburia inulinivorans in a subject suffering from or at risk of muscle loss and/or reduced muscular strength due to myotonia.

Amyotrophic lateral sclerosis (ALS) refers to a series of pathological changes caused by motor neuron damage. The pathological changes include motor neuron degeneration, gliosis, nerve fibre abnormality, loss of myelinated fibres in the corticospinal tract and anterior root of spinal nerve. The manifestations of medullary motor neuron damage, for example, include facial muscle, language and swallowing dysfunction; the manifestations of spinal cord motor neuron damage include muscle spasm, muscle weakness, muscle atrophy, paralysis, and respiratory failure. ALS is characterized by progressive manifestations of dysfunction of the lower and upper motor neurons. Lower motor neurons connect the brainstem and spinal cord to muscle fibres, and their dysfunction leads to muscle atrophy, spasm, and fasciculation. Upper motor neurons originate from the motor area of the cerebral cortex or brainstem, and carry motor information to the motor neurons that directly respond to stimulate the target muscle. Their dysfunction leads to spasm (continuous muscle contractions that interfere with gait, movement, and speech) and pathological reflexes. ALS can be divided into sporadic ALS (sALS) and familial ALS (fALS) according to whether it has familial inheritance. There is no ALS family history for sporadic ALS patients, and there is more than one ALS patient in the family with familial ALS. According to the different ways of inheritance, familial ALS can be divided into autosomal dominant inheritance, autosomal recessive inheritance, and X chromosome linked inheritance. Motor nerves can nourish muscle tissues. After cutting off the motor nerve, in the muscle the glycogen synthesis slows down, the protein decomposition accelerates, and the muscle gradually shrinks.

In some examples, the Roseburia inulinivorans or compositions thereof are for use in treating or preventing or for use in methods of treating or preventing muscle loss and/or reduced muscular strength in subjects suffering from or at risk of ALS. In some examples, there is provided methods of increasing the population of Roseburia inulinivorans in a subject suffering from or at risk of muscle loss and/or reduced muscular strength due to ALS.

Spinal muscular atrophy refers to a group of clinically and genetically diverse diseases in which symmetrical muscle weakness or loss occurs due to degeneration or loss of anterior horn cells or brainstem nuclei in the spinal cord. Spinal muscular atrophy (SMA) caused by damage to the nerves that control muscles is an example of a neurodegenerative disease. In eukaryotes, SMN may be an autosomal recessive disorder caused by mutations in the SMN1 gene encoding the SMN (survival motor neuron) protein.

In some examples, the Roseburia inulinivorans or compositions thereof are for use in treating or preventing or for use in methods of treating or preventing muscle loss and/or reduced muscular strength in subjects suffering from or at risk of SMA. In some examples, there is provided methods of increasing the population of Roseburia inulinivorans in a subject suffering from or at risk of muscle loss and/or reduced muscular strength due to SMA.

Cachexia or marasmus is characterized by extreme thinness, especially caused by muscle loss, caused by prolonged illness or inadequate calorie or protein intake. Cachexia may be associated with cancer, infection (for example by HIV or AIDS), renal failure, heart failure, autoimmunity, and drug or alcohol addiction. Therefore, there is a reason for increasing muscle mass and/or muscular strength in such subjects.

In some examples, the Roseburia inulinivorans or compositions thereof are for use in treating or preventing or for use in methods of treating or preventing muscle loss and/or reduced muscular strength in subjects suffering from or at risk of cachexia. In some examples, there is provided methods of increasing the population of Roseburia inulinivorans in a subject suffering from or at risk of muscle loss and/or reduced muscular strength due to cachexia.

Age-related muscle wasting or sarcopenia, is the progressive loss of muscle mass and muscle strength that occurs with age. This condition is thought to be a consequence of decreased muscle synthesis and repair in addition to increased muscle breakdown. In age-related muscle wasting the bundles of muscle fibres can shrink because individual fibres are lost. Furthermore, due to disuse muscle atrophy in such subjects, muscle fibres also get smaller. Treatments may reverse this muscle atrophy. Sarcopenia, also lead to changes in the type of muscle fibres. Type 1 and Type 2 decrease with aging at a similar rate, whereas Type 2 muscle fibre thickness does not change much but Type 1 muscle fibre thickness decreases significantly with sarcopenia.

Age-related muscle wasting begins at middle age and accelerates throughout the remainder of life. The most commonly used definition for the condition is appendicular skeletal mass/body height² (kg/m²) less than two standard deviations below the mean value for young adults. This disorder can lead to decreased mobility, functional disability and loss of independence.

The term “elderly” as used herein, refers to a subject of at least 40 years of age, including at least 45 years of age, at least 50 years of age, at least 55 years of age, at least 60 years of age, at least 65 years of age, at least 70 years of age, at least 75 years of age, and including at least 80 years of age or greater. The term “elderly” also includes the groups of from 45 years of age to 100 years of age, and the group of from 55 years of age to 80 years of age.

In some examples, the Roseburia inulinivorans or compositions thereof are for use in treating or preventing or for use in methods of treating or preventing muscle loss and/or reduced muscular strength in subjects suffering from or at risk of sarcopenia. In some examples, there is provided methods of increasing the population of Roseburia inulinivorans in a subject suffering from or at risk of muscle loss and/or reduced muscular strength due to sarcopenia.

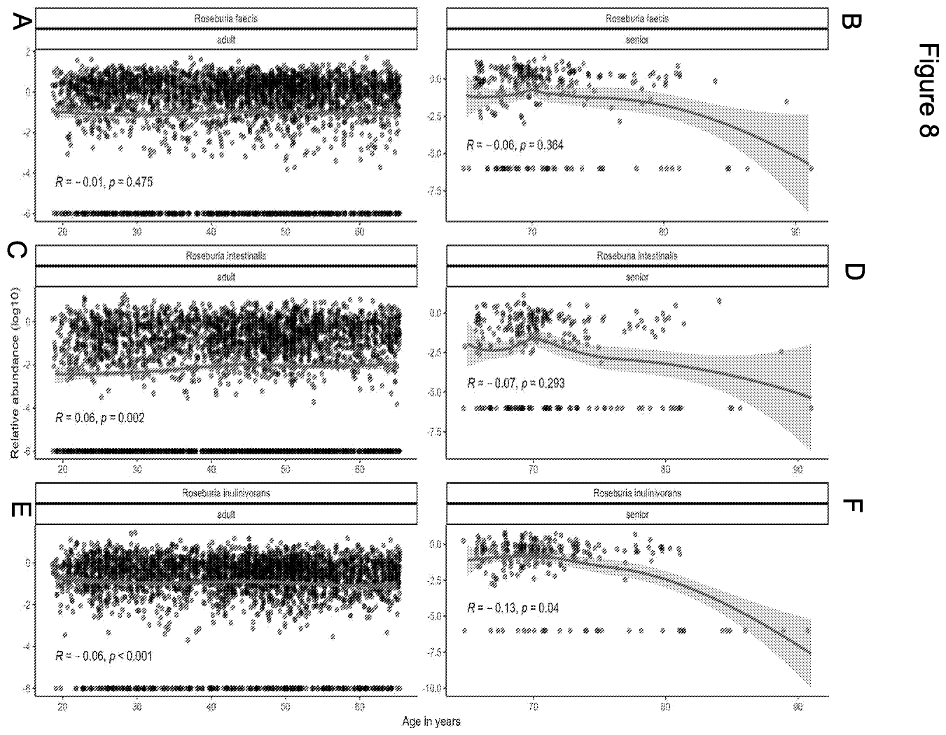

As shown in FIG. 8, Roseburia inulinivorans population decreases with increasing age. Therefore, in some examples, the Roseburia inulinivorans or compositions thereof as disclosed herein are for use in treating or preventing or for use in methods of treating or preventing muscle loss and/or reduced muscular strength due to aging. In some examples, the Roseburia inulinivorans or compositions thereof as disclosed herein are for use in treating or preventing or for use in methods of treating or preventing ageing.

Acardiotrophy is a disease in which the heart becomes atrophied by an external or internal factor, which may show brownish atrophy where myocardial fibers become dry and thin which can be found in starvation, wasting diseases, and senility.

In some examples, the Roseburia inulinivorans or compositions thereof are for use in treating or preventing or for use in methods of treating or preventing acardiotrophy. In some examples, there is provided methods of increasing the population of Roseburia inulinivorans in a subject suffering from or at risk of acardiotrophy.

Frailty is defined as a clinically recognizable state of increased vulnerability resulting from aging-associated decline in reserve and function across multiple physiologic systems such that the ability to cope with every day or acute stressors is compromised. In the absence of an established quantitative standard, frailty has been operationally defined by Fried et al. as meeting three out of five phenotypic criteria indicating compromised energetics: (1) weakness (grip strength in the lowest 20% of population at baseline, adjusted for gender and body mass index), (2) poor endurance and energy (self-reported exhaustion associated with VO₂ max), (3) slowness (lowest 20% of population at baseline, based on time to walk 15 feet, adjusting for gender and standing height), (4) low physical activity (weighted score of kilocalories expended per week at baseline, lowest quintile of physical activity identified for each gender; e.g., less than 383 kcal/week for males and less than 270 kcal/week for females), and/or unintentional weight loss (10 lbs. in past year). For example, see Fried L P, Tangen C M, Walston J, et al., “Frailty in older adults: evidence for a phenotype.” J. Gerontol. A. Biol. Sci. Med. Sci. 56(3):M146-M156 (2001). A pre-frail stage, in which one or two of these criteria are present, identifies a high risk of progressing to frailty.

In some examples, the Roseburia inulinivorans or compositions thereof are for use in treating or preventing or for use in methods of treating or preventing muscle loss and/or reduced muscular strength in subjects suffering from or at risk of frailty. In some examples, there is provided methods of increasing the population of Roseburia inulinivorans in a subject suffering from or at risk of muscle loss and/or reduced muscular strength due to frailty.

Examples of trauma or injury to muscle, bone or nerve that may lead to reduced muscle strength and/or muscle mass include damage to muscle of a subject as a result of an accident sports trauma, endocrine disorder, disease, trauma, or surgical procedure. For example, bone breakages, ligament injuries, muscle sprain or muscle strain. In some examples, injury may include skeletal muscle injury such as ruptures, tears or bruising of skeletal muscle induced by eccentric muscle contractions, elongations and/or muscle overload.

Examples of trauma or injury include hip fractures and acute knee injuries. Patients with hip fractures often have muscle atrophy prior to fracture and muscle wasting is a key contributor to hip fracture in many patients. Following hip fracture, muscle and strength is lost due to disuse, and often hip fracture patients do not return to pre-fracture levels of ambulation or function. Furthermore, many hip fracture patients are also afflicted with conditions such as COPD, ESRD and cancer, which can contribute to significant muscle wasting and predispose them to hip fracture. Therefore, patients may be treated with Roseburia inulinivorans described herein if they are at risk of hip fracture. There is considerable therapeutic urgency associated with hip fracture patients since these patients must be operated on immediately. Therefore, post-operative treatment with Roseburia inulinivorans as described herein can help aid the recovery of hip fracture patients by diminishing the loss of muscle mass and strength, and/or improving the recovery of muscle mass and strength. A subject at risk of hip fracture or a subject with a hip fracture may be treated with Roseburia inulinivorans or by a method that increases Roseburia inulinivorans as described herein prior to surgery.

In some examples a subject may be treated to increase the population of Roseburia inulinivorans prior to any surgery in order to increase muscle mass and/or muscular strength in order to compensate for loss of muscle mass and/or strength due to the surgery or during the recovery period.

In some examples, the Roseburia inulinivorans or compositions thereof are for use in treating or preventing or for use in methods of treating or preventing muscle loss and/or reduced muscular strength in subjects suffering from trauma or injury. In some examples, there is provided methods of increasing the population of Roseburia inulinivorans in a subject suffering from or at risk of muscle loss and/or reduced muscular strength due to trauma or injury.

Cancer patients often display muscle wasting which can lead to hospitalization, infection, dehydration, hip fracture, and ultimately death. For example, a 10% loss of muscle mass can be associated with a dramatically inferior prognosis of the cancer patient.

A significant portion of cancer patients suffer from weight loss due to progressive atrophy of adipose tissue and muscle wasting. It is estimated that about 20% of cancer deaths are caused by muscle loss. Muscle wasting is generally a good predictor of mortality in many diseases conditions. Data from research on AIDS, starvation and cancer indicate that loss of more than 30-40% of individual pre-illness lean body mass is fatal (for example, see DeWye W D. In Clinics in Oncology. Edited by Calman K C and Fearon K C H. London: Saunders, 1986, Vol. 5, no 2, p. 251-261; Kotter D P, et al. 1990 J Parent Enteral Nutr 14:454-358; and Wigmore S J, et al. 1997 Br J Cancer 75:106-109). Thus, the possible mitigation of muscle atrophy through increasing the population of Roseburia inulinivorans may be helpful in improving quality of life in subjects suffering from cancer as well as extending life expectancy. Treatment with Roseburia inulinivorans or the methods for increasing the population of Roseburia inulinivorans as described herein may improve the performance status of the cancer patient, for example to allow full chemotherapy or a more aggressive use of chemotherapy, and to improve patient quality of life.

Cancer includes, for example, pancreatic ductal adenocarcinoma; pancreatic cancer; breast cancer; melanoma; non-small cell lung cancer; small cell lung cancer; nasopharyngeal cancer; hepatocellular cancer; colorectal cancer; oesophageal cancer; gastric cancer; anal cancer; small intestine cancer; mesothelioma; kidney cancer; renal cell carcinoma; bladder cancer; prostate cancer; ovarian cancer; vulval cancer; cervical cancer; penile cancer; uveal melanoma; retinoblastoma; sarcoma; osteosarcoma; glioblastoma; adrenocortical carcinoma; neuroblastoma; Wilms tumour; endometrial cancer; and thyroid cancer.

In some examples, the Roseburia inulinivorans or compositions thereof are for use in treating or preventing or for use in methods of treating or preventing muscle loss and/or reduced muscular strength in subjects suffering from cancer. In some examples, there is provided methods of increasing the population of Roseburia inulinivorans in a subject suffering from or at risk of muscle loss and/or reduced muscular strength due to cancer.

In some examples, the subject may be or have been immobilized for an extended period of time due to sedation, hospital stay (for example in intensive care) or due to being in a coma. The loss of muscle mass is one of the most important and frequent problems observed in patients hospitalized in the ICU, affecting the cardiovascular, the respiratory and the circulatory system. It is mainly caused by the prolonged bed rest, sedation and immobilization.

In some examples, the Roseburia inulinivorans or compositions thereof are for use in treating or preventing or for use in methods of treating or preventing muscle loss and/or reduced muscular strength in critically ill, sedated or comatose subjects. In some examples, there is provided methods of increasing the population of Roseburia inulinivorans in a subject suffering from or at risk of muscle loss and/or reduced muscular strength due to critical illness, sedation or coma.

The terms “treatment” and “treating” should be taken as encompassing therapy undertaken in order to prevent, slow down, or reduce an undesired physiological change or disorder, such as the loss of muscle mass and/or muscle strength. Accordingly, “treatment” refers to both therapeutic treatment and prophylactic or preventative measures, wherein the object is to prevent or slow down (lessen) the loss of muscle mass and/or muscle strength. In other words, terms “treatment,” “treat,” and “treating” refer to reversing, alleviating, delaying the onset of, or inhibiting the progress of e.g. the loss of muscle mass and/or muscle strength.

For purposes of the present invention, beneficial or desired results include, but are not limited to, alleviation of symptoms, diminishment of extent of the loss of muscle mass and/or muscle strength, stabilized state of muscle mass and/or muscle strength (which is to say, muscle mass and/or muscle strength is not worsening), delay or slowing of the loss of muscle mass and/or muscle strength, amelioration or palliation of the loss of muscle mass and/or muscle strength (either partially or totally).

Treatment may bring about prolonged survival as compared to expected survival if not receiving treatment. Alternatively, or additionally, treatment may provide a subject with an improved standard of life as compared to that which would be expected if not receiving treatment.

The therapeutic uses and methods of treatment of the present invention may be of particular benefit in preventing or reducing the loss of muscle mass and/or muscle strength in subjects receiving treatment.

The methods and therapeutic uses of the invention may be administered in combination with other suitable treatments, either concurrently or sequentially. Other treatments that subjects may receive may depend on the underlying cause of loss of muscle mass and/or muscle strength.

In addition, Roseburia inulinivorans or compositions thereof may be administered in combination with other probiotics that may enhance or supplement the effects described herein. In some examples, the methods of increasing the population of Roseburia inulinivorans may include administering one or more prebiotics each of which increase the populations of other beneficial microbiota in the subject.

For example, the prebiotic may be a prebiotic as described herein. For example, the prebiotic may include one or more of inulin, melibiose, glucose, sucrose, starch, acetate, lactate, butyrate, propionate, fructo-oligosaccharides, fucose and/or methionine. Therefore, in some examples there is provided a prebiotic composition for use in treating or preventing or for use in methods of treating or preventing muscle loss and/or reduced muscular strength in a subject as described herein. For example for use in treating or preventing muscle loss and/or reduced muscular strength in subjects suffering from or at risk of muscular atrophy. For example for use in treating or preventing muscle loss and/or reduced muscular strength in subjects suffering from or at risk of muscular dystrophy. For example for use in treating or preventing muscle loss and/or reduced muscular strength in subjects suffering from or at risk of myasthenia. For example for use in treating or preventing muscle loss and/or reduced muscular strength in subjects suffering from or at risk of myotonia. For example for use in treating or preventing muscle loss and/or reduced muscular strength in subjects suffering from or at risk of ALS. For example for use in treating or preventing muscle loss and/or reduced muscular strength in subjects suffering from or at risk of SMA. For example for use in treating or preventing muscle loss and/or reduced muscular strength in subjects suffering from or at risk of cachexia. For example for use in treating or preventing muscle loss and/or reduced muscular strength in subjects suffering from or at risk of sarcopenia. For example for use in treating or preventing muscle loss and/or reduced muscular strength in subject suffering from or at risk of acardiotrophy. For example for use in treating or preventing muscle loss and/or reduced muscular strength in subjects suffering from or at risk of frailty. For example for use in treating or preventing muscle loss and/or reduced muscular strength in subjects suffering from trauma or injury. For example for use in treating or preventing muscle loss and/or reduced muscular strength in subjects suffering from cancer. For example for use in treating or preventing muscle loss and/or reduced muscular strength in critically ill, sedated or comatose subjects.

The subject may be referred to herein as a patient. The terms “subject”, “individual”, and “patient” are used herein interchangeably. As used herein, the term “subject” is intended to include humans and animals. Typically, a subject refers to a human or animal that, within their body, has a cell deficient in homologous recombination. Examples of subjects include mammals, e.g., humans, dogs, cows, horses, pigs, sheep, goats, cats, mice, rabbits, rats, and transgenic non-human animals. In some examples, subjects include companion animals, e.g. dogs, cats, rabbits, and rats. In some examples, subjects include livestock, e.g., cows, pigs, sheep, goats, and rabbits. In some examples, subjects include thoroughbred or show animals, e.g. horses, pigs, cows, and rabbits.

### Non-Therapeutic Uses

Given the effects that the methods and compositions described herein may have on a subject's muscular strength and/or muscle mass, there is also provided methods of improving a subject's muscular strength and/or muscle mass for cosmetic purposes.

In such examples, the subject may not suffer from a disease, condition, or disorder as described herein in which a loss of muscle mass and/or muscle strength occurs, but an increase of muscle mass and/or strength is desired (e.g., in an animal subject to increase meat production, or in a human subject to increase muscle mass and/or strength to improve athletic ability or improve physical appearance).

In some examples, the methods and compositions described herein may be for the non-therapeutic use of maintaining the ability to perform an activity, or for reducing a deterioration in the ability to perform an activity. Such activities may be day to day activities such as walking, climbing stairs or other common activities undertaken by a subject in the standard course of a day. For example, light household work (dusting, washing dishes), heavy household work (washing windows, washing floors), shopping, rising from a chair, sitting in a chair, outdoor gardening, or paid work activities. In some examples, the subject may be a healthy elderly subject. The subject may not have reduced muscle mass and/or strength. Alternatively, the subject may have reduced muscle mass and/or strength due to natural ageing but not a degree that is of clinical significance (i.e. does not suffer from muscular atrophy or sarcopenia) or caused by a pathological condition.

In some examples, the methods and compositions described herein may be for the non-therapeutic use of improving or maintaining athletic training, performance or recovery by a subject. In such examples, the subject may be an athlete. The term “athlete” may refer to a subject who is proficient in sports and other forms of physical exercise or a subject who is trained or skilled in exercises, sports, or games requiring physical strength, agility, endurance, speed or stamina or a subject who possesses above average physical skills such as strength, agility, endurance, speed or stamina and is suited for physical competition.

“Improving or maintaining athletic training, performance or recovery” refers to a comparison of athletic training, performance or recovery using objective factors for a particular individual. Such objective factors may include ability to continue a particular exercise or training event over a given period of time. Such exercises or training events are known to those of skill in the art and can be determined based on the particular athletic event or sport engaged in by the subject. Such objective recovery factors include the length of time it takes for an individual to perform at the same or similar level between an athletic event, training, performance or physical activity.

As used herein, “athletic performance” refers to attaining improved physical performance (i.e. physical fitness) from engaging in particular types and frequencies of exercise. This encompasses cardiovascular fitness, body composition fitness and the overall condition of subject's musculature as well as athletic ability. “Body composition” refers to the percentages of fat, muscle, water and bone in a subject's body. “Athletic ability” refers to the success level of performing physical tasks e.g. running fast, jumping high, running for a long time, lifting heavy weights. For example, an athletic performance trait may be exercise endurance, muscular power, muscle damage and/or injury risk.

The terms “improving athletic performance”, “enhancing athletic performance” or any variations thereof as used herein should be understood to encompass improvement of at least one sport parameter. Non-limiting examples of such improvements are: testosterone elevation, cortisol reduction, fatigue reduction, faster recovery from exercise, reduced muscle soreness, improved endurance, improved muscle strength, and improved muscle size. Improving athletic performance may include elongating the duration of the physical activities, performing it faster and combinations thereof. Improving athletic performance may include improving endurance, fatigue reduction, faster recovery and any combination thereof. The terms “improving athletic performance” refers to a change in athletic performance, where the change is defined as a difference in the performance between a subject obtaining the administration according to the invention and a subject in similar conditions or the same subject who does not. In some embodiments, the change is detected or measured on the same subject. The change is regarded as an improvement if such change is positive for said subject. Athletic performance exact definition depends on the type of athletic activity.

As such, the methods and compositions described herein may be for the non-therapeutic use of improving a subject's body composition or physical appearance. In some examples, the methods and compositions described herein may alter subject's body mass index, for example increase or decrease a subject's BMI depending on the outcome desired by the subject.

The term “improved recovery” or variations thereof encompass one or more of general recovery following exercise, muscle recovery, heart and/or lung recovery, recovery of gas concentrations in the blood system, mental recovery, restoration of energy, reduction of physical and mental stress, metabolic recovery, immediate recovery, short-term recovery and training recovery The term “muscle recovery” refers to restoring the muscle condition to its original condition before the exercise or to a better condition. As such, “improving muscle recovery” refers to reducing or shortening the time required for muscle recovery. Measurable parameters that can indicate a reduction in muscle recovery time may be the reduction of the lactate levels in the blood along time.

It should be clear that improvement of athletic training, performance or recovery in professional athletes may be small if measured as a percent of change but are significant in their absolute value. The rate of improvement of athletic training, performance or recovery of non-professional athletes may be much more significant. The methods and compositions described herein may improve athletic training, performance or recovery by at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% in comparison to a reference, as determined under comparable conditions. Athletic training, performance or recovery may be reflected in any measurable values such as speed (of running or swimming), weight (raised), number of sets performed, distance (a person can jump, run or swim), and time the athlete may keep performing the exercise etc.

In some examples, there is provided a prebiotic composition for increasing Roseburia inulinivorans in methods of improving or maintaining athletic training, performance or recovery by a subject and/or improving muscular strength, muscle mass, and/or pulmonary capacity in a subject for non-therapeutic purposes. For example, the prebiotic may be a prebiotic as described herein. For example, the prebiotic may include one or more of inulin, melibiose, glucose, sucrose, starch, acetate, lactate, butyrate, propionate, fructo-oligosaccharides, fucose and/or methionine. Therefore, in some examples there is provided a prebiotic composition for

In some examples, the methods and compositions described herein may be used in livestock to increase muscle mass in order to increase the yield of meat from the livestock. Animals suitable for meat production include, but are not limited to cows, bulls, bison, horses, sheep, goats, pigs, ducks, geese, lamas, camels, dromedary, boars, turkeys, and chickens.

Aspects of the invention are demonstrated by the following non-limiting examples.

### EXAMPLES

### Materials and Methods

### Human Experiments

A total of 90 (64 women) young healthy adults, aged 18-25 years old, were included in the study. This study used the baseline data of the ACTIBATE study¹³, an exercise-based randomized controlled trial (Clinical Trials.gov ID: NCT02365129). All assessments were performed in Granada (Spain) between October and November in 2016. The participants reported to perform less than 20 min moderate-vigorous physical activity on <3 days/week, had a stable body weight over the last 3 months (<3 kg change), did not smoke, did not taking any meditation (including antibiotics in the last 3 months), did not presenting any acute or chronic illness and were not pregnant. The study protocol and the written informed consent were performed in accordance with the Declaration of Helsinki, as revised in 2013, and were approved by the Human Research Ethics Committee of the University of Granada (n° 924) and that of the “Servicio Andaluz de Salud” (Centro de Granada, CEI-Granada).

### Muscular Strength Measurements

Handgrip strength test, and the 1 repetition maximum (1-RM) leg and bench press tests were used for measuring muscular strength capacity of the participants.

Handgrip strength test was determined through a Takei 5401 digital Grip-D hand dynamometer (Takei, Tokyo, Japan)¹⁴, with results expressed in kilograms. The participants were asked for squeeze the grip gradually and continuously, and they were encouraged to do their best while performing the tests. The test was performed twice, alternating between both hands with a 1 min rest between efforts. Men executed the test with grip span of the dynamometer fixed at 5.5 cm, however for women, it was adjusted to the individual's hand size, according to a validated equation¹⁴. A KEISER® Air 300 pneumatic resistance machine and KEISER® Power rack (Keiser, Fresno, CA, USA) were used for performing 1-RM leg and bench press tests, respectively, with the aim of assessing lower and upper body strength. The Wathen equation was used for estimating 1-RM values, because the participants were sedentary¹⁵. The participants lifted the maximum weight they could lift a maximum number of 1-10 times in both tests [1-RM=Weight lifted per repetition (kg)/((48.8+53.8e^{-0.075×number of repetitions})/100)]. They had three attempts to provide the needed lift data, moreover if they failed to do it, they could return another day to repeat the test. At the beginning, several lifts with no weight were conducted for participants to familiarize with the exercise. The participants were able to perform more than 10 repetitions with a particular weight, they stopped and rested for 5 min and next they followed with another try with a heavier weight. The test was finished when participants made <10 repetitions at their maximum strength capacity. Data for all muscular strength tests were saved in absolute terms. Participants were categorized into tertiles according to their handgrip strength levels (i.e., low 19.25-27.50 kg; medium 27.55-32.20 kg and high 32.55-54.05 kg).

### Cardiorespiratory Fitness

Before carrying out a cardiorespiratory fitness test, the participants had to fulfil specific requirements: fasting for 3-5 h, not to perform vigorous exercise in the previous 48 h neither moderate exercise in the previous 24 h, and not to consume coffee or tea in the latter period.

The cardiorespiratory fitness test was performed using a maximum treadmill exercise (H/P/Cosmos Pulsar treadmill, H/P/Cosmos Sports & Medical GmbH, Nussdorf-Traunstein, Germany), following the modified Balke protocol¹³. The warm-up consisted in walking of 1 min at 3 km/h, followed by 2 min at 4 km/h. The incremental protocol started at the fourth minute, at a speed of 5.3 km/h (0% slope). The speed was kept constant while every minute the treadmill slope was increased by 1% until the participants became exhausted. Finally, they went through a cooling-down period at 4 km/h (0% slope) for 5 min. During the test, respiratory gas exchange (oxygen volume (VO₂) and carbon dioxide volume (VCO₂)) was monitored by indirect calorimetry using a CPX Ultima CardioO₂ gas exchange analysis system (Medical Graphics Corp, St Paul, MN, USA), with a facemask, model 7400 plastic (Hans Rudolph Inc., Kansas City, MO, USA) and equipped with a preVent™ metabolic flow sensor (Medical graphics Corp, St Paul, MN, USA)¹⁶. VO₂ was measured using a galvanic fuel cell, whereas VCO₂ using a non-dispersive infra-red sensor¹⁶. The criteria for defining maximum oxygen volume (VO₂ₘₐₓ) were a respiratory exchange ratio of ≥1.1, a VO₂ plateau (changes of <100 ml/min over three consecutive 10 s intervals), and a heart rate within 10 beats/min of the age-predicted maximum (209−0.73×age)¹⁷. Time to exhaustion was measured in seconds. VO₂ₘₐₓ was represented in absolute terms. Participants were categorized into tertiles according to their VO₂ₘₐₓ capacity (i.e., low 794-2528 mL/min, intermediate 2544-3077 mL/min and high 3102-4811 mL/min).

### Body Composition Assessment

Participants' weight and height were measured, without shoes and wearing the standard clothes, using SECA scale and stadiometer (model 799, Electronic Column Scale, Hamburg, Germany). Body mass index (BMI) was calculated as body weight (kg)/body height (m²). Body composition was evaluated by Dual Energy X-ray Absorptiometry (DEXA, HOLOGIC, Discovery Wi, Marlborough, MA).

### Stool Collection and DNA Extraction

The participants collected approximately 50 g of fecal sample in plastic sterile containers, which it was transported in portable cooler to research centre. Fecal samples were stored at −80° C. until extraction of DNA. QIAamp DNA Stool Mini Kit (QIAGEN, Barcelona, Spain) was used for extraction of DNA, following manufacturer's instructions. The samples were incubated at 95° C. to ensure lysis of both Gram-positive and Gram-negative bacteria. Then, DNA was quantified with a NanoDrop ND1000 spectrophotometer (Thermo Fisher Scientific, DE, USA). Finally, DNA purity was determined by measuring the ration of absorbance at A260/280 nm (protein contamination¹⁸) and A260/230 nm (salt and phenol contamination¹⁹)

### Sequencing Analysis

DNA extracted was amplified by polymerase chain reaction (PCR) by primer pairs, 16S Amplicon PCR Forward Primer: 5′CCTACGGGNGGCWGCAG 3′ (SEQ ID NO: 1), and 16S Amplicon PCR Reverse Primer: 5′GACTACHVGGGTATCTAATCC 3′ (SEQ ID NO: 2) targeting the V3 and V4 hypervariable regions of the bacterial 16S rRNA gene²⁰. All PCRs were executed in 25 μL reaction volumes incorporating 12.5 μL 2×KAPA HiFi Hotstart ready mix (KAPA Biosystems, Woburn, MA, USA), 5 μL of each forward and reverse primers (1 μM) and 2.5 μL of extracted DNA (10 ng) under the following cycling circumstances: (a) denaturation at 95° C. for 3 min, (b) cycles of denaturation at 95° C. for 30 s, (c) annealing at 55° C. for 30 sec, (d) elongation at 72° C. for 30 sec, (e) a final extension at 72° C. for 5 min.

To purify the 16S V3 and V4 amplicon away from free primers and primer dimer AMPure XP beads (Beckman Coulter, Indianapolis, IN, USA) were used. Next, the index PCR attached dual indices and Illumina sequencing adapters using the Nextera XT Index Kit (Illumina, San Diego, CA, USA), on a thermal cycler using the requirements previously mentioned. After, AMPure XP beads (Beckman Coulter, Indianapolis, IN, USA) were used for purifying of the pooled PCR products. The resultant amplicons were sequenced at MiSeq (Illumina, USA), using paired-end (2×300 nt) Illumina MiSeq sequencing system (Illumina, San Diego, CA, USA).

### Bioinformatics Analysis

The FastQ files were analysed with “dada2”²¹ package in R software²², and got 11,659,014 paired-ends with an average of 126,728±33,395 reads per sample. Cut-off 10,000 reads was surpassed for all samples. Samples were resampled to an equal sequencing depth of 30,982 reads using “phyloseq”²³ package in R²², coming back 11,158 phylotypes. The “classifier” function from the Ribosomal Database Project (RDP) was used for assigning the taxonomic affiliation of phylotypes, based on the naïve Bayesian classification²⁴ with a pseudo-bootstrap threshold of 80%. A total of 209 genuses were obtained that belong to 16 different phyla. “seqmatch”²⁵ function from RDP was performed to define the discriminatory power of each sequence read with the purpose of annotating species assignments. Annotation was executed according to criteria published previously²⁶, the annotation was chosen with threshold of 297% of coincidence. Microbial communities were analysed at different taxonomic levels (phylum to species), calculating relative abundances, expressed as percentages. In the feces of these participants, it was possible to identify the Roseburia faecis M72/1 (DSM 16840), Roseburia intestinalis L1-82 (DSM 14610) and Roseburia inulinivorans A2-194 (DSM 16841) species.

### Culture and Preparation of Roseburia Species for the Mouse Experiment

Roseburia faecis M72/1 (DSM 16840), Roseburia intestinalis L1-82 (DSM 14610) and Roseburia inulinivorans A2-194 (DSM 16841) were obtained from the Leibniz Institute DSMZ (Deutsche Sammlung von Mikroorganismen and Zellkulturen GmbH, Wolfenbuttel, Germany). All Roseburia species were cultured in a Baker Ruskinn Concept 1000 anaerobic cabinet on YCFA medium (pH6.8)³¹ solidified with 1.5% bacteriological agar or in liquid YCFAC medium (Anaerobe Systems) as described²⁷⁻³⁰.

For preparation of inocula, a single colony from YCFA agar was inoculated into liquid medium, and incubated for 16 h at 37° C. The resulting overnight culture was diluted into fresh prereduced YCFAC medium and incubated until the culture reached mid-exponential growth phase. Then, 50 mL aliquots of culture were centrifuged in parafilm sealed tubes at 5000×g for 20 min and the culture supernatant was removed. Pellets were resuspended in 2.8 mL prereduced phosphate buffered saline (PBS)-carbonate buffer, pooled, and glycerol was added to 15% (vol/vol). 1 mL aliquots of bacterial suspension were made in 3 mL syringes (BD) that were sealed with a Luer lock combi lock and parafilm and stored immediately at −80° C. With the exception of the centrifugation and storage steps, the procedure was performed inside the anaerobic cabinet. The procedure was designed to minimize loss of viability due to exposure to oxygen.

The number of viable bacteria administered to mice was calculated by plating the bacterial culture before, during and after preparation for administration to mice. Identity of the isolates was confirmed using 16S rRNA sequencing and MALDI Biotyper (Bruker) at multiple points during the preparation (data not shown).

### Animal Experiments

A total amount of thirty-two 6-week-old male mice (C57BL/6J background, Charles River Laboratories) were group-housed at room temperature (22° C.) and 12:12 hour light-dark cycle with ad libitum access to water and standard laboratory chow diet (Rat and Mouse No. 3 Breeding, SDS, Horley, United Kingdom). During the first two weeks all mice received a cocktail of antibiotics (200 μL; vancomycine 0.25 mg/mL, metronidazole 0.5 mg/mL, neomycin 0.5 mg/mL and ampicillin 0.5 mg/mL) diluted in sterile water by oral gavage every day to deplete their original gut bacteria. After these 2 weeks, mice were randomized to one of the 4 treatment groups (n=8; each group): i) mice treated with 200 μL of PBS+15% glycerol (control mice), ii) mice treated with Roseburia faecis M72/1 (200 μL; 10\hat{ }9 CFU/mL), iii) mice treated with Roseburia intestinalis L1-82 (200 μL; 10\hat{ }9 CFU/mL) and iv) mice treated with Roseburia inulinivorans A2-194 (200 μL; 10\hat{ }9 CFU/mL). Vehicle control and bacterial suspensions were transplanted into mice via oral gavage 3 times per week for a total period of 8 weeks. For this purpose, previously produced aliquots were allowed to thaw and were kept sealed on ice until the moment of gavage. The animal experiments were carried out according to the Institute for Laboratory Animal Research Guide for the Care and Use of Laboratory Animals, and was approved by the National Committee for Animal Experiments (Protocol No. 11600202010187) and by the Ethics Committee on Animal Care and Experimentation of the Leiden University Medical Center (Protocol No. PE.18.063.005). All animal procedures conformed to the guidelines from Directive 2010/63/EU of the European Parliament on the protection of animals used for scientific purposes.

### Muscular Strength Measurement

Forelimb grip strength was measured 2 weeks after the treatment with antibiotics (0 week), 4 weeks, 6 weeks and 8 weeks after the treatment with different bacteria. Forelimb grip strength was assessed by means of a grip strength meter (Columbus Instruments, USA). Mice were tested five times, with three consecutive measurements per trial (15 in total), and a two minutes interval between trials. The three highest measured values were averaged to calculate the absolute strength, which was divided by the body weight in grams.

### Body Weight and Body Composition

Body weight was measured with a scale, and body composition was measured in conscious mice using an EchoMRI-100 analyzer weekly (EchoMRl, Houston, TX).

### Quantification of Short-Chain Fatty Acids and Amino Acids Levels in the Caecum

In the isolated caecum content of the mice, short-chain fatty acids (SCFAs) and amino acids were analyzed using gas chromatography-mass spectrometry (GC-MS) with slight modifications to a previously established protocol⁴⁵. In this study, 10 μL of caecum diluted with PBS was transferred to a glass vial, along with a mixture comprising 250 μL of acetone, 10 μL of a 1 ppm internal standards solution, and an additional 10 μL of ethanol (all from Sigma Aldrich). The samples were subjected to derivatization using pentafluorobenzyl bromide (PFBBr) to facilitate the analysis. Specifically, 100 μL of 172 mM PFBBr (Thermo) in acetone was added to the samples, which were then thoroughly mixed and incubated at a temperature of 60° C. for 30 minutes. Following cooling to room temperature, a liquid-liquid extraction step was performed, employing 500 μL of n-hexane (Sigma-Aldrich) and 250 μL of GC-MS grade water. The upper organic layer was subsequently transferred to a fresh glass vial for subsequent GC-MS analysis. Calibration standards were prepared similarly, except that plasma was not added, and instead, 10 μL of ethanol was substituted with 10 μL of a standard solution (Sigma-Aldrich) in ethanol. The GC-MS analysis was conducted using a Bruker Scion 436 GC instrument equipped with an Agilent VF-5 ms capillary column (25 m×0.25 mm i.d., 0.25 μm film thickness), coupled to a Bruker Scion TQ MS system. Sample injection was achieved utilizing a CTC PAL autosampler (G6501-CTC), where a splitless injection of 1 μL of the sample was performed at a temperature of 280° C. Helium (99.9990%) served as the carrier gas at a constant flow rate of 1.20 mL/min. The GC temperature program encompassed an initial 1-minute isothermal hold at 50° C., followed by a linear ramp of 40° C./min to 60° C., maintaining this temperature for 3 minutes. Subsequently, there was a linear increase at 25° C./min to 200° C., followed by another linear increase of 40° C./min to 315° C., maintaining this final temperature for 2 minutes. The transfer line and ionization source were maintained at 280° C. Methane (99.9990%) was employed as the chemical ionization gas, with a pressure set to 15 psi. The analysis was performed in the selected ion monitoring mode, detecting negatively charged ions.

### Untargeted Metabolomics in Plasma and Muscle Tissue

Plasma and gastrocnemius muscle were shipped to Beijing Genomic Institute (BGI Inc, Beijing, China) for performing untargeted metabolomics. In short, BGI preserved the samples, and performed metabolite extraction and bioinformatic analyses. Firstly, samples that had been stored at −20° C. were thawed in a 4° C. refrigerator until no visible ice remained. 100 μL of each sample, including the quality control (QC) samples, were added to individual Eppendorf tubes, and the remaining samples were kept frozen. A solution containing the internal standard (methanol:acetonitrile:water=4:2:1, v/v/v) was added (700 μL) to each sample (including quality control samples), followed by shaking for 1 minute and placement in a −20° C. freezer for 2 hours. The samples were centrifuged at 25,000 g and 4° C. for 15 minutes. The supernatants (600 μL) were transferred to new Eppendorf tubes. Drying was performed using a drying machine. A mixture of acetonitrile and pure water (7:3 v/v) was added (100 μL) to reconstitute the samples, which were vortexed for 10 minutes until pellets were fully dissolved. The reconstituted samples were centrifuged again at 25,000 g and 4° C. for 15 minutes, and supernatants again transferred to new Eppendorf tubes. Secondly, after importing the off-line data of MS into compound discoverer 3.2 (Thermo Fisher Scientific, USA) software and analyzing the MS data in combination with bmdb (BGI metabolome database), mzcloud database and chemspider online database, a data matrix containing information such as metabolite peak area and identification results were obtained. Thirdly, in the data post-processing step, Probabilistic Quotient Normalization (PQN) was used to normalize the data and obtain relative peak areas. Quality control-based robust LOESS signal correction (QC-RLSC) was employed to correct batch effects and removed metabolites with high variability in their relative peak areas. PQN involved obtaining an overall reference vector and correcting the actual sample based on the reference vector, while QC-RLSC used local polynomial regression fitting to correct experimental sample signals using QC samples. Quality control was assessed using Principal Component Analysis (PCA), a statistical method for analyzing multidimensional data. Log transformation and Pareto scaling were applied to compute principal components, and the resulting PCA plot was used to observe sample group separation and identify abnormal samples. The variability within and between groups was also examined. The overall analysis involved the functional annotation of metabolites using databases such as the Human Metabolome Database and KEGG PATHWAY. These annotations helped to understand the properties and metabolic pathways associated with the identified metabolites. Partial Least Squares-Discriminant Analysis (PLS-DA) was utilized to screen for differences between groups. This supervised method established a model between metabolite expression and sample categories to predict sample categories. A PLS-DA model was established between the comparative analysis groups, and log 2 transformation was applied with Pareto scaling and cross-validation for model validation.

### Determination of Muscle Fiber Size

From the gastrocnemius muscles of the mice, transverse serial cross-sections (7 μm thick) were obtained using a cryostat (Microm HM 505 N Cryostat Microtome) maintained at −25° C. and were mounted onto glass microscope slides. Cryosections were stained with hematoxylin/eosin solution for measuring the cross-sectional area and with NADH (0.5 Mm) for fiber typing using established histological techniques. Morphometry of musculoskeletal fiber was performed on a Leica DM18 microscopy using a Leica MC170 HD camera, controlled by the LAS-X program. Images were taken at 10× magnification and mosaics of the cuts were made to obtain large images. All muscle fiber of the sections were quantified (an average of 850 fibers per sample) and their cross-sectional area was expressed in μm² using ImageJ software. Fiber typing was performed by determining the area covered by type I fiber, as stained with the Nitrotetrazolium Blue chloride in dark blue (representing the NADH activity), and assuming the remaining fiber corresponding to type II fiber, by using ImageJ software.

### Access to Publicly Available Data Sets and Data Analyses

To validate findings on a larger scale, the inventors leveraged the curated MetagenomicData (CMD) resource (v3.6.2). This valuable resource provides taxonomic and functional profiles of more than 21,000 fecal metagenomes, which are publicly accessible to researchers and the general public alike. To ensure the robustness of our analysis, several filtering steps were applied to the metagenomic samples. Samples from individuals in the ‘newborn’ age category and those taken while under antibiotic treatment were excluded. Additionally, samples lacking exact age information in years were removed, and in cases where multiple samples were available from the same individual, only one sample was randomly selected. Furthermore, only samples from individuals classified as ‘healthy’ in the CMD resource were retained. As a result, a total of 3,809 stool metagenomes from healthy individuals were obtained. Focusing on the adult (19-64 years of age) and senior (65+) age categories, which were of primary interest, the data was further filtered to include only samples from these specific age groups. This yielded 3,257 metagenomes from the adult category and 255 metagenomes from the senior category. To address the issue of zero values in log space, a pseudocount of 1e-6 to all relative abundances was added. Subsequently, correlations and p-values using Spearman correlations within the stat_cor function were completed. Additionally, to visually depict the data, locally weighted scatterplot smoothing (LOESS) was applied to obtain a smooth curve fit.

### Results

In the cohort of 90 inactive young adults (on average 22 years old) it was observed that the relative abundance of Roseburia inulinivorans-DSM16841 within feces was 2.2-fold higher in individuals with high handgrip strength, bench press strength and leg press strength compared to individuals with low muscular strength (Table 1).

TABLE 1
Tertiles of muscular strength in 90 young adults

| | Low | | | Medium | | | High | | | |
|---|---|---|---|---|---|---|---|---|---|---|
| Genus (%) | (N = 30) | | | (N = 30) | | | (N = 30) | | | P |
| Roseburia faecis-DSM16840 | 0.36 | ± | 0.72 | 0.30 | ± | 0.65 | 0.31 | ± | 0.41 | 0.402 |
| Roseburia intestinalis- | 0.24 | ± | 0.57 | 0.24 | ± | 0.48 | 0.67 | ± | 1.07 | 0.009 |
| DSM14610 | | | | | | | | | | |
| Roseburia inulinivorans- | 0.77* | ± | 1.27 | 0.95 | ± | 1.51 | 1.66* | ± | 1.78 | 0.015 |
| DSM16841 | | | | | | | | | | |

Data are presented as mean ± standard deviations (SD). P value significant <0.05.
*Equal symbols mean significant different between groups.

In addition, it was found that individuals with high pulmonary capacity in response to exercise also had 2.6-fold higher relative abundance of Roseburia inulinivorans-DSM16841 compared to individuals with low pulmonary capacity (Table 2).

TABLE 2
Tertiles of pulmonary capacity in response to exercise test (VO₂max)
in 90 young adults

| | Low | | | Medium | | | High | | | |
|---|---|---|---|---|---|---|---|---|---|---|
| Genus (%) | (N = 30) | | | (N = 30) | | | (N = 30) | | | P |
| Roseburia faecis- | 0.36 | ± | 0.74 | 0.36 | ± | 0.63 | 0.26 | ± | 0.39 | 0.409 |
| DSM16840 | | | | | | | | | | |
| Roseburia intestinalis- | 0.14 | ± | 0.25 | 0.48 | ± | 0.13 | 0.51 | ± | 0.57 | 0.018 |
| DSM14610 | | | | | | | | | | |
| Roseburia inulinivorans- | 0.50*† | ± | 1.02 | 1.51* | ± | 1.88 | 1.31† | ± | 1.54 | 0.004 |
| DSM16841 | | | | | | | | | | |

Data are presented as mean ± standard deviations (SD).
VO₂max: maximum volume of oxygen.
*,†Equal symbols mean significant different between groups.

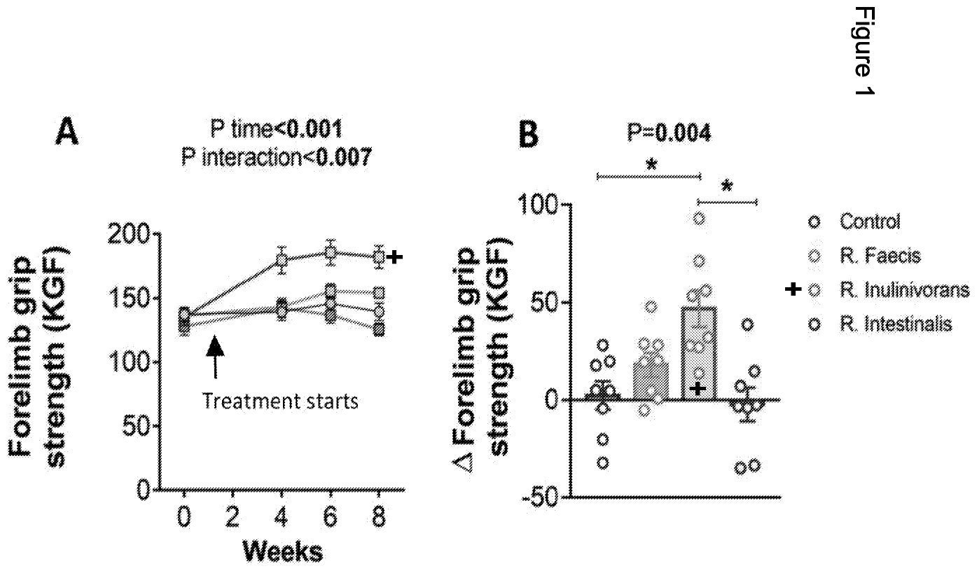

Since the cross-sectional nature of the human study precluded establishing the direction of these relationships, a healthy and young group of male C57Bl/6J mice were treated with Roseburia inulinivorans-DSM16841, whereas other groups of mice were treated with Roseburia faecis-DSM16840 and Roseburia intestinalis-DSM14610 as controls. Roseburia inulinivorans-DSM16841 increased the forelimb grip strength in mice compared to the other groups in the absence of exercise training by approximately 30% (FIG. 1).

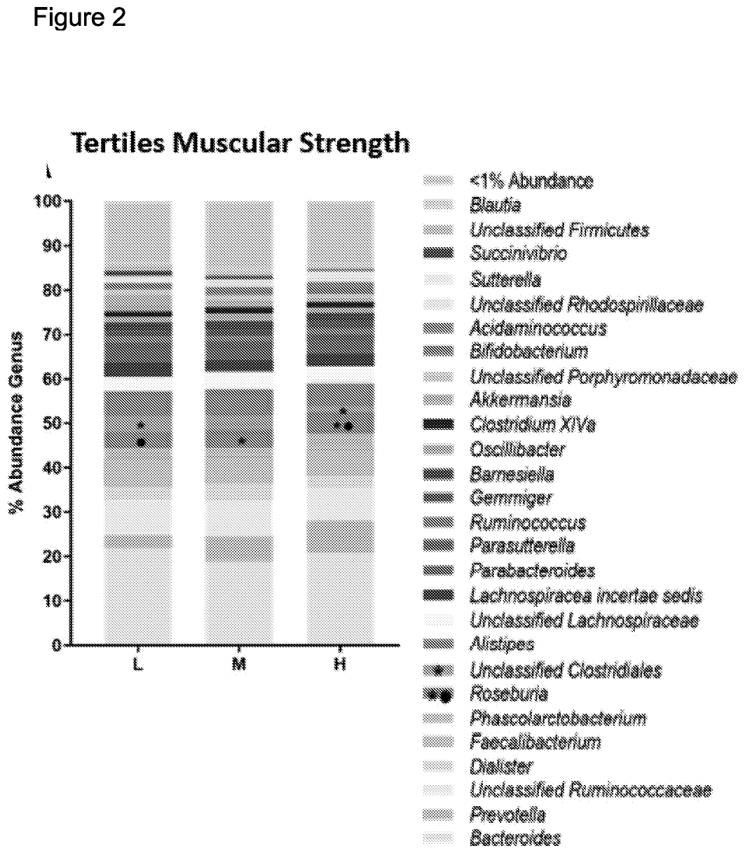

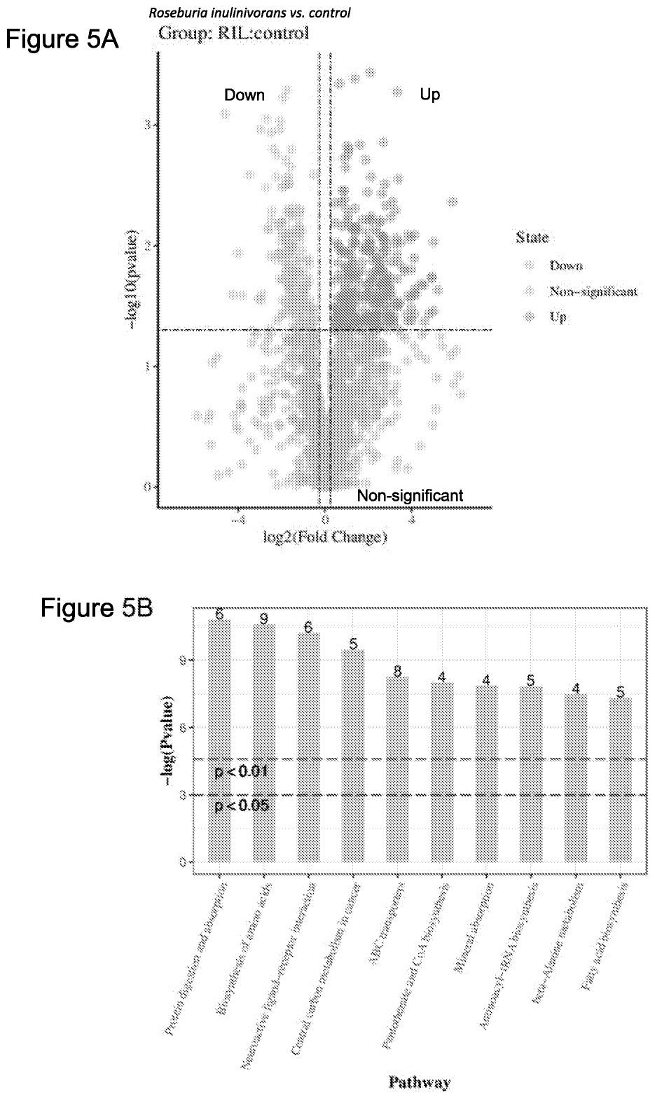

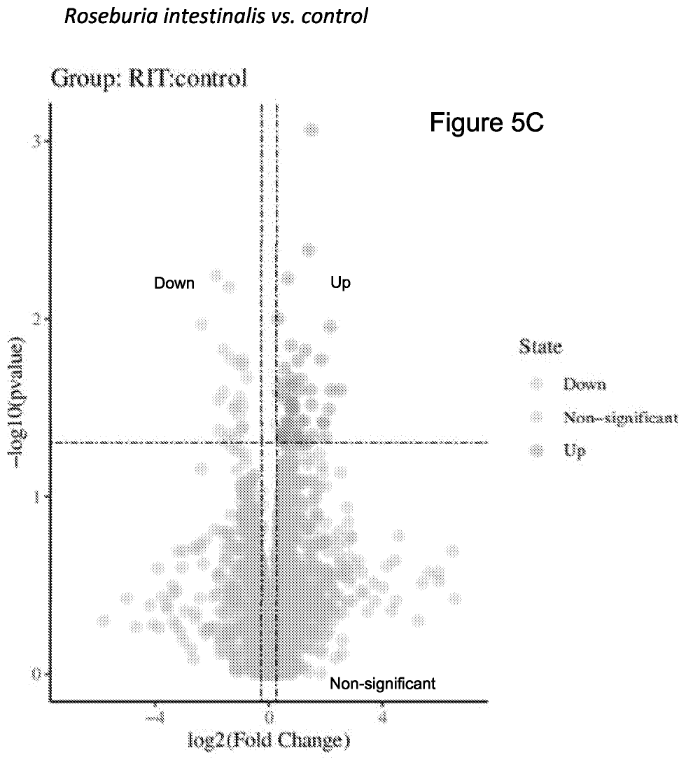

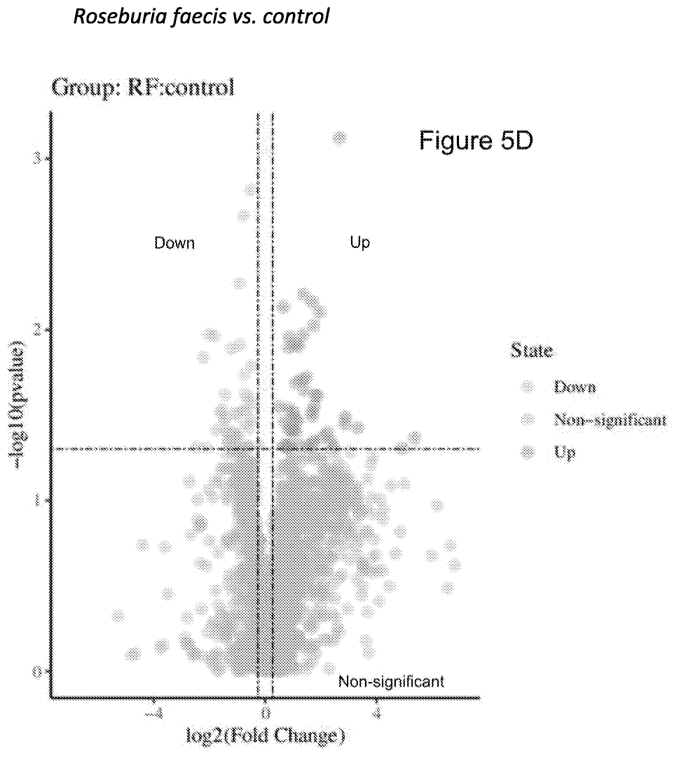

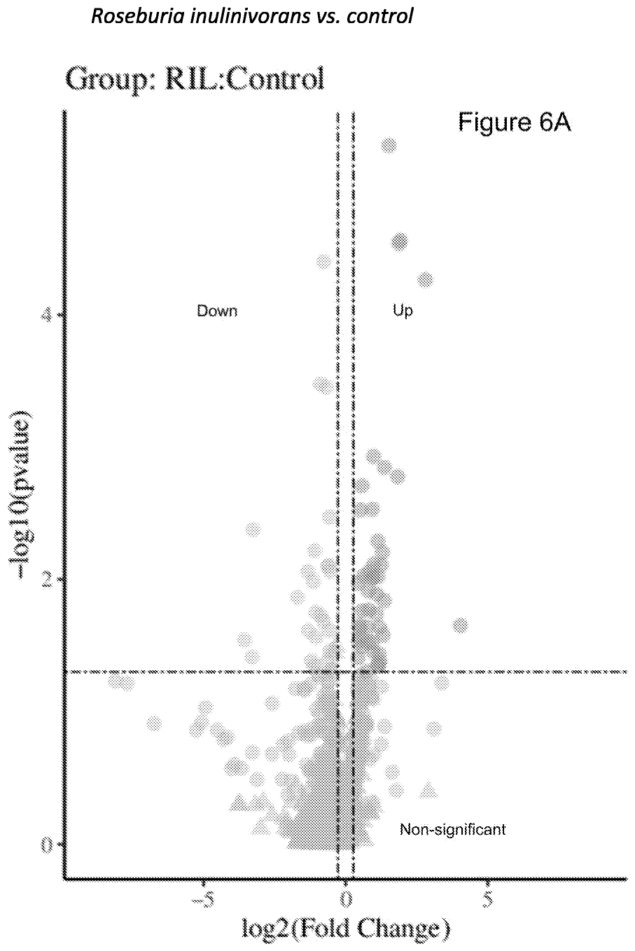

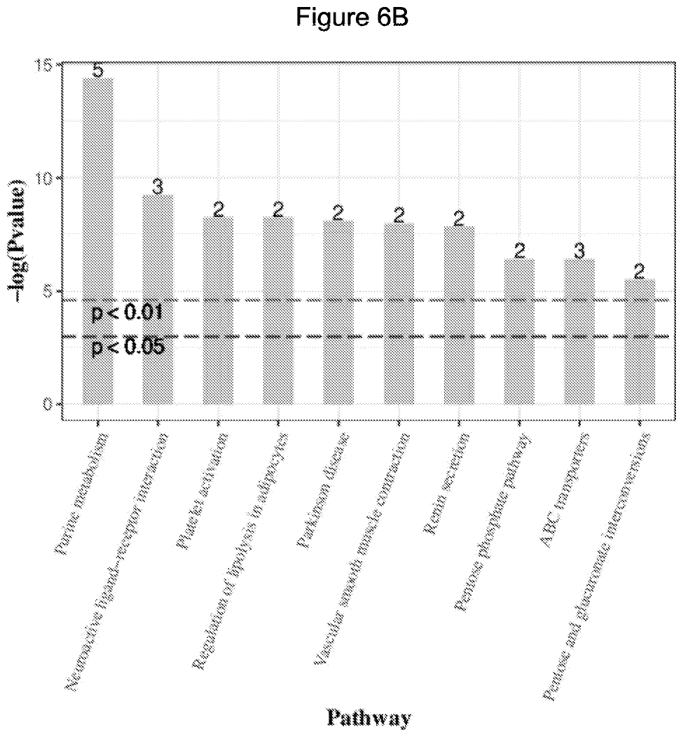

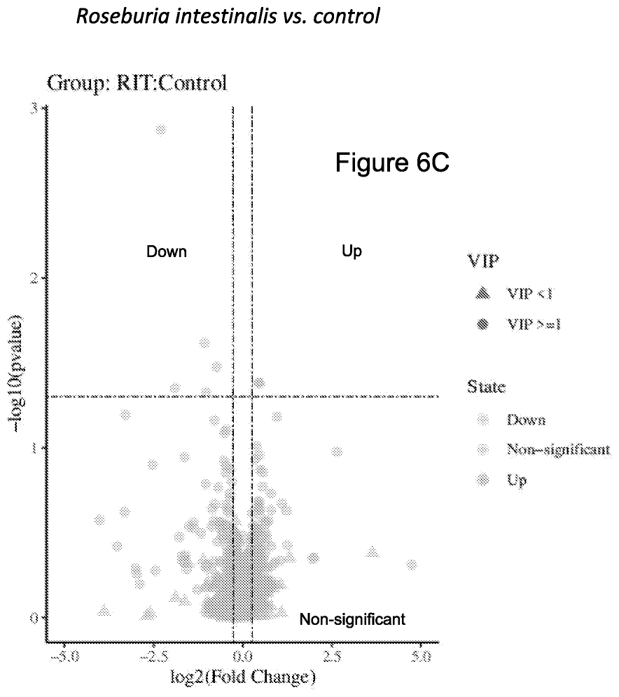

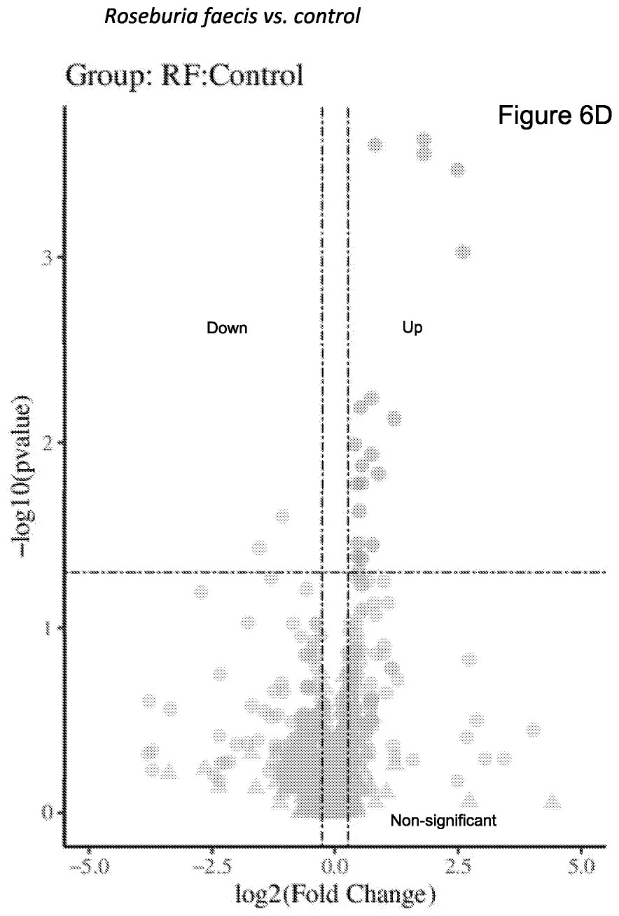

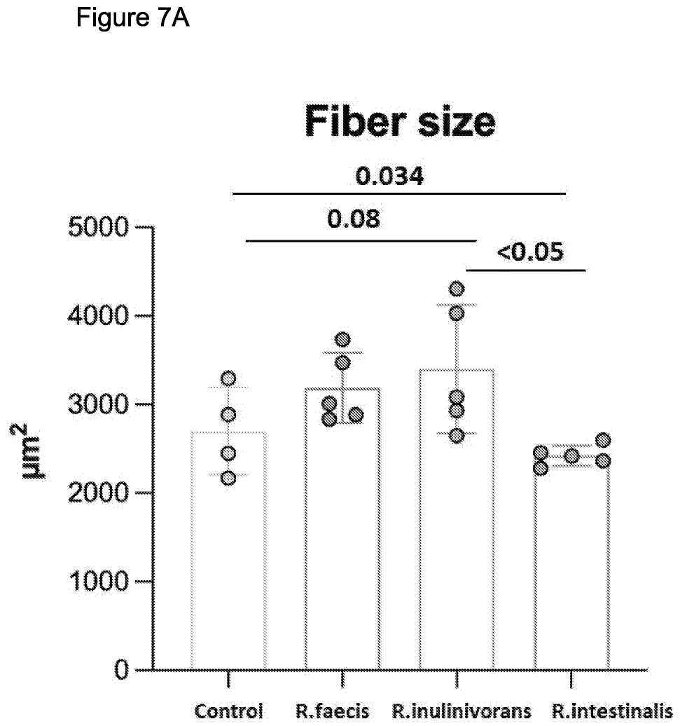

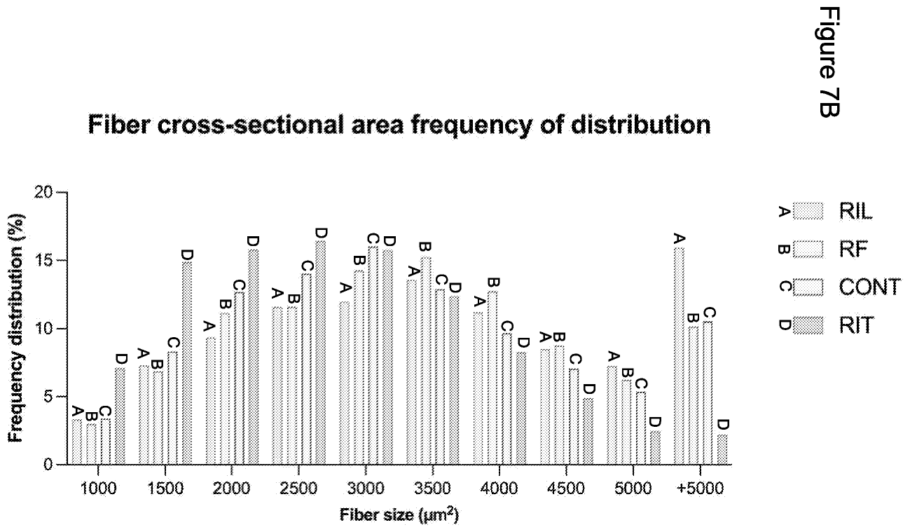

Without any need for exercise training, R. inulinivorans highly increased the forelimb grip strength, while the other treatments were ineffective (FIG. 1). These effects are probably mediated via high production of amino acids by R. Inulinivorans, which enhances the balance between protein anabolism vs. protein catabolism in the skeletal muscle. Roseburia genus in the gut associates to higher muscular strength (FIG. 2). R. inulinivorans induced a profound modification in the plasma metabolome compared to the control group, while R. intestinalis and R. faecis only had modest effects. R. inulinivorans specifically upregulated pathways associated with protein digestion and absorption, amino acid biosynthesis, neuroactive ligand-receptor interaction, central carbon metabolism, ABC transporters, pantothenate and CoA biosynthesis, mineral absorption, aminoacyl-tRNA biosynthesis, beta-alanine metabolism, and fatty acid metabolism (FIG. 5A to 5D). In the gastrocnemius muscle, R. inulinivorans profoundly modified pathways linked to purine metabolism, neuroactive ligand-receptor interaction, platelet activation, regulation of lipolysis, and the pentose phosphate pathway compared to the control group, while R. intestinalis and R. faecis had only modest effects (FIG. 6A to 6D). R. inulinivorans tended to enhance muscle fiber size compared to the control group (P=0.08) and significantly increased muscle fiber size in the gastrocnemius compared to mice treated with R. intestinalis (P<0.05) but not R. faecis (FIGS. 7A and 7B).

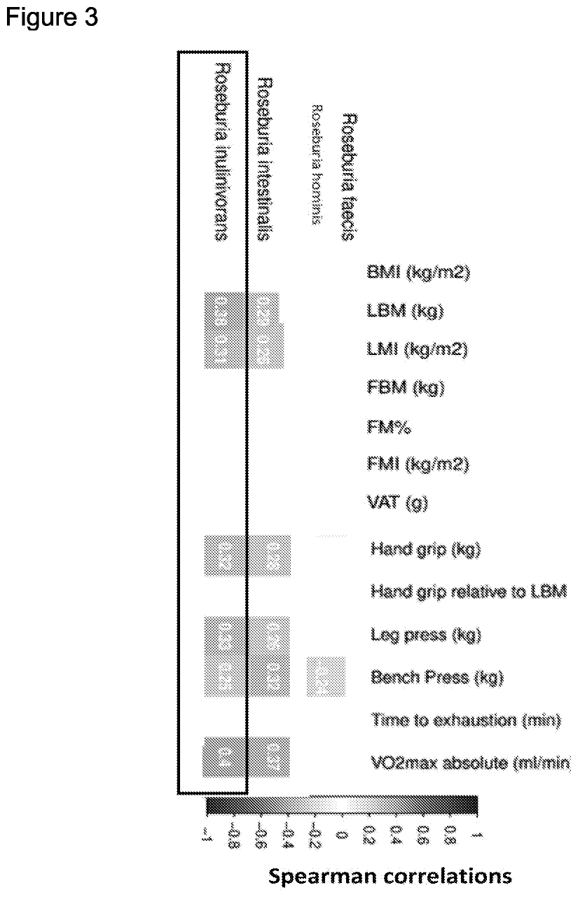

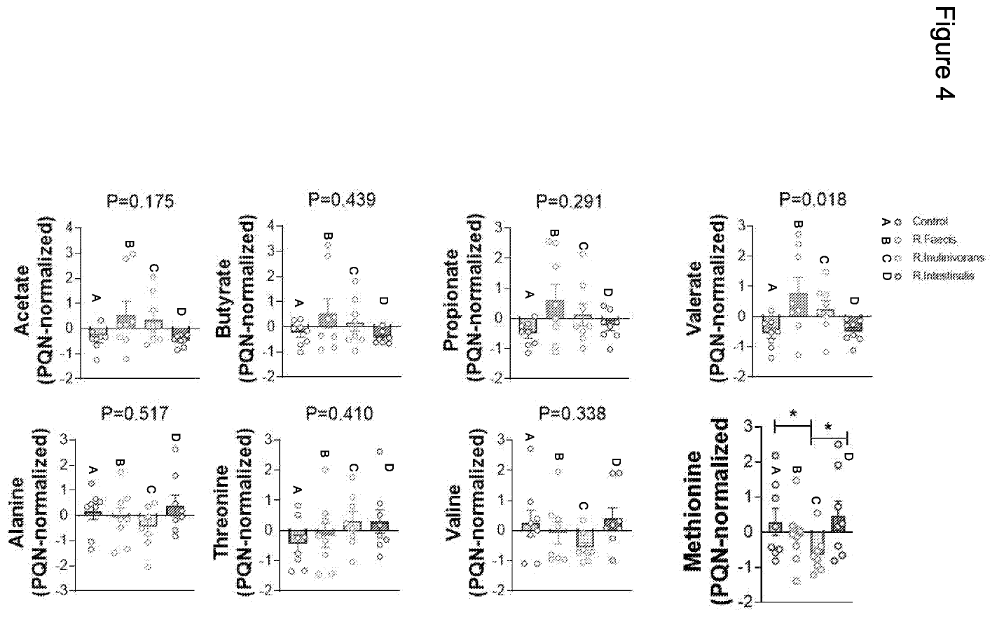

Deeper analyses showed that Roseburia inulinivorans were the main species driving the associations with physical fitness outcomes (FIG. 3). Physical fitness levels strongly depend on the muscle mass of the individuals, and Roseburia inulinivorans was positively related with the amount of muscle mass of subjects (FIG. 3).

Sarcopenia is a degenerative condition characterized by age-related loss of muscle mass and strength. Publicly available datasets were utilised to investigate potential changes in the relative abundance of three specific Roseburia strains employed in the mouse experiment throughout the lifespan. It was discovered that the relative abundance of Roseburia inulinivorans showed a negative correlation with age in adults (age range: 20 to 65 years old; R=−0.06, P<0.001, FIG. 8E). Moreover, in the senior population (age range: 65 to 92 years old), it was observed that a similar negative correlation between the relative abundance of Roseburia inulinivorans and age (R=−0.13, P<0.001, FIG. 8F). None of the other strains showed negative correlations with age in the adult or senior groups (FIGS. 8A-D), showing the effect being specific for Roseburia inulinivorans.

### DISCUSSION

A very clear bidirectional link exists between physical exercise and the composition of microorganisms that live in the intestine, the so called gut microbiota. Exercise training reduces the presence of harmful bacteria via unknown mechanisms³². However, little is known about how the bacterial community can improve a human's exercise capacity.

Very recently, it has been shown that oral gavage with Veillonella atypica, a bacterium that is present in the human gut, improves 10% the running capacity of mice³³ This bacterium is capable of consuming lactate, which is massively produced during aerobic exercise, and producing short chain fatty acids (i.e., acetate and propionate) that enhance muscular mitochondrial function¹⁸. In another study, high-fat diet-fed mice received a daily oral gavage with 16 different bacteria, mainly Lactobacillus and Bifidobacterium species, for 4 weeks, which decreased body fat (+40%), running capacity (+20%) and forelimb grip strength (+20%) compared to a control group³⁴. However, whether these bacteria can be used for restoring muscular strength in muscle atrophy models remained unaddressed until very recently.

In a recent study, mice received an oral gavage with Akkermansia muciniphila and Faecalibacterium prausnitzii for 5 weeks, while during the last week of treatment their hindlimbs were cast to induce muscle atrophy³⁵. It was observed that mice receiving Akkermansia muciniphila or Faecalibacterium prausnitzii did not lose hindlimb grip strength compared to mice that were not cast, indicating that these bacteria can preserve muscular strength. There is no evidence suggesting that the presence of any of the aforementioned bacteria are related to higher muscular strength or muscle mass in humans. On top of that, understanding the underlying mechanisms that explain how these bacteria improve muscular strength is important for developing future interventions to palliate human muscle loss.

The data provided herein shows that young individuals (n=30) with high muscular strength and high pulmonary capacity (i.e., VO₂max) showed significantly higher relative abundance of Roseburia inulinivorans in their feces than individuals with medium (n=30) or low (n=30) muscular strength and pulmonary capacity.

In severe sarcopenia, Roseburia inulinivorans is undetectable in feces³⁶. Similarly, the relative abundance of the Roseburia genus is diminished in patients with anorexia nervosa³⁷ and in children with cerebral palsy³⁸, of whom muscle mass is substantially reduced. Additionally, data from publicly available sources show that the relative abundance in feces of Roseburia inulinivorans declines across the lifespan, while no significant changes are observed for Roseburia faecis or Roseburia intestinalis. This observation suggests that as humans age and experience muscle mass loss, the relative abundance of this particular strain also decreases. Since the cross-sectional nature of all these observations precluded establishing causality, an experimental study in C57Bl/6J mice was performed. Without any need for exercise training, 8 weeks of oral gavage with Roseburia inulinivorans highly increased the forelimb grip strength, while treatments with other bacteria or vehicle were ineffective. Targeted metabolomics of cecal content revealed that mice treated with Roseburia inulinivorans had unaffected short chain fatty acid (SCFA) levels but decreased methionine and increased aspartate levels, which may provide an as yet unaddressed underlying mechanism. Although Roseburia inulinivorans is a well-known butyrate-producing bacterium, it expresses enzymes involved in aspartate and methionine metabolism³⁹. Supplementation of aspartate is commonly used in the field of exercise physiology for delaying fatigue during exercise⁴⁰ or increasing muscle fat oxidation⁴¹. However, nothing is known about effects of aspartate supplementation on gut microbiota composition or the relative abundance of the Roseburia genus. Methionine restriction diet increases the relative abundance of Roseburia genus in mice⁴² and improves running capacity via activation of the GCN2/ATFG4-dependent pathway in muscles⁴³, as well as increases skeletal muscle mass in old mice⁴⁴. Therefore, it is plausible that the low methionine levels create a favourable environment for boosting the intestinal growth of Roseburia inulinivorans to produce higher amounts of aspartate that enhances muscle metabolism resulting in an improvement in muscular strength. These amino acids are taken up by enterocytes and reach the liver via the portal vein. Considering the significant alterations observed in the plasma metabolome, it is reasonable to hypothesize that the liver metabolizes the amino acids derived from R. inulinivorans, which induces notable increases in e.g., glutamate or aspartate in the circulation. Glutamate and aspartate are essential amino acids for purine metabolism, which is the primary pathway affected in the gastrocnemius muscles of mice treated with R. inulinivorans. Interestingly, stimulation of the rapamycin complex 1 (mTORC1) pathway appeared crucial for increasing muscular strength and muscle fiber size through strength training⁴⁶. Recent studies have also indicated that mTORC1 activation generates precursors for synthesizing purines and increases muscle fiber size⁴⁷. Therefore, the increase in muscular strength induced by R. inulinivorans may be attributed to an activation of the purine metabolism pathway, which may increase muscle fiber size, suggesting a potential mechanism for its effects. Altogether, the data provide evidence that Roseburia inulinivorans improves muscular strength via modulation of specific amino acids through an undisclosed and innovative physiological mechanism.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Sequences

5′CCTACGGGNGGCWGCAG 3′ (SEQ ID NO: 1)
5′GACTACHVGGGTATCTAATCC 3′ (SEQ ID NO: 2)

### REFERENCES

1. Garcia-Hermoso A, Cavero-Redondo I, Ramirez-Velez R, et al. Muscular Strength as a Predictor of All-Cause Mortality in an Apparently Healthy Population: A Systematic Review and Meta-Analysis of Data From Approximately 2 Million Men and Women. Archives of Physical Medicine and Rehabilitation. 2018; 99(10):2100-2113.e5. doi:10.1016/j.apmr.2018.01.008
2. Ortega F B, Silventoinen K, Tynelius P, Rasmussen F. Muscular strength in male adolescents and premature death: Cohort study of one million participants. BMJ (Online). 2012; 345(7884):1-12. doi:10.1136/bmj.e7279
3. Theadom A, Rodrigues M, Roxburgh R, et al. Prevalence of muscular dystrophies: A systematic literature review. Neuroepidemiology. 2014; 43(3-4):259-268. doi:10.1159/000369343
4. Bull F C, AI-Ansari S S, Biddle S, et al. World Health Organization 2020 guidelines on physical activity and sedentary behaviour. British Journal of Sports Medicine. 2020; 54(24):1451-1462. doi:10.1 136/bjsports-2020-102955
5. Scheiman J, Luber J M, Chavkin T A, et al. Meta-omics analysis of elite athletes identifies a performance-enhancing microbe that functions via lactate metabolism. Nature Medicine. June 2019. doi:10.1038/s41591-019-0485-4
6. Plovier H, Everard A, Druart C, et al. A purified membrane protein from Akkermansia muciniphila or the pasteurized bacterium improves metabolism in obese and diabetic mice. Nature Medicine. 2017; 23(1):107-113. doi:10.1038/nm.4236
7. Duncan S H, Aminov R I, Scott K P, Louis P, Stanton T B, Flint H J. Proposal of Roseburia faecis sp. nov., Roseburia hominis sp. nov. and Roseburia inulinivorans sp. nov., based on isolates from human faeces. International Journal of Systematic and Evolutionary Microbiology. 2006; 56(10):2437-2441. doi:10.1099/ijs.0.64098-0
8. Lahiri S, Kim H, Garcia-Perez I, et al. The gut microbiota influences skeletal muscle mass and function in mice. Science Translational Medicine. 2019; 11(502). doi:10.1126/scitranslmed.aan5662
9. Borgo F, Riva A, Benetti A, et al. Microbiota in anorexia nervosa: The triangle between bacterial species, metabolites and psychological tests. PLoS ONE. 2017; 12(6):1-17.doi:10.1371/journal.pone.0179739
10. Huang C, Li Y, Feng X, et al. Distinct Gut Microbiota Composition and Functional Category in Children With Cerebral Palsy and Epilepsy. Frontiers in Pediatrics. 2019; 7(October):1-8. doi:10.3389/fped.2019.00394
11. Ticinesi A, Mancabelli L, Tagliaferri S, et al. The gut-muscle axis in older subjects with low muscle mass and performance: a proof of concept study exploring fecal microbiota composition and function with shotgun metagenomics sequencing. International Journal of Molecular Sciences. 2020; 21(23):1-16. doi:10.3390/ijms21238946
12. Fielding R A, Reeves A R, Jasuja R, Liu C, Barrett B B, Lustgarten M S. Muscle strength is increased in mice that are colonized with microbiota from high-functioning older adults. Experimental Gerontology. 2019; 127(July):110722. doi:10.1016/j.exger.2019.110722
13. Sanchez-Delgado, G. et al. Activating brown adipose tissue through exercise (ACTIBATE) in young adults: Rationale, design and methodology. Contemporary Clinical Trials 45, 416-425 (2015).
14. Ruiz-Ruiz, J., Mesa, J. L. M., Gutierrez, A. & Castillo, M. J. Hand size influences optimal grip span in women but not in men. The Journal of Hand Surgery 27, 897-901 (2002).
15. Wood, T. M., Maddalozzo, G. F. & Harter, R. A. Accuracy of seven equations for predicting 1-RM performance of apparently healthy, sedentary older adults. Measurement in Physical Education and Exercise Science 6, 67-94 (2002).
16. Sanchez-Delgado, G. et al. Reliability of resting metabolic rate measurements in young adults: Impact of methods for data analysis. Clin Nutr (2017) doi:10.1016/j.clnu.2017.07.026.
17. Midgley, A. W., McNaughton, L. R., Polman, R. & Marchant, D. Criteria for determination of maximal oxygen uptake: A brief critique and recommendations for future research. Sports Medicine vol. 37 1019-1028 (2007).
18. Hassan, R. et al. Guidelines for nucleic acid detection and analysis in hematological disorders. Malays J Pathol 37, 165-73 (2015).
19. Lucena-Aguilar, G. et al. DNA Source Selection for Downstream Applications Based on DNA Quality Indicators Analysis. Biopreservation and Biobanking 14, 264-270 (2016).
20. Pr Herlemann, D. et al. Transitions in bacterial communities along the 2000 km salinity gradient of the Baltic Sea. The ISME Journal 5, 1571-1579 (2011).
21. Callahan, B. J. et al. DADA2: High resolution sample inference from Illumina amplicon data. Nature Methods 13, 581-583 (2016).
22. R Core Team. R: A Language and Environment for Statistical Computing. (2019). WO 2024/0254185′ PCT/NL2023/050404
23. McMurdie, P. J. & Holmes, S. phyloseq: An R Package for Reproducible Interactive Analysis and Graphics of Microbiome Census Data. PLoS ONE 8, e61217 (2013).
24. Wang, Q., Garrity, G. M., Tiedje, J. M. & Cole, J. R. Naïve Bayesian Classifier for Rapid Assignment of rRNA Sequences into the New Bacterial Taxonomy. APPLIED AND ENVIRONMENTAL MICROBIOLOGY 73, 5261-5267 (2007).
25. Cole, J. R. et al. Ribosomal Database Project: data and tools for high throughput rRNA analysis. Nucleic Acids Research 42, D633-D642 (2014).
26. Schulz, C. et al. The active bacterial assemblages of the upper GI tract in individuals with and without Helicobacter infection. doi:10.1136/gutjnl-2016-312904.
27. Stanton, T. B., Duncan, S. H. & Flint, H. J. Roseburia. in Bergey's Manual of Systematics of Archaea and Bacteria 1-7 (Wiley, 2015). doi:10.1002/9781118960608.gbm00652.
28. Duncan, S. H. et al. Proposal of Roseburia faecis sp. nov., Roseburia hominis sp. nov. and Roseburia inulinivorans sp. nov., based on isolates from human faeces. International Journal of Systematic and Evolutionary Microbiology 56, 2437-2441 (2006).
29. Duncan, S. H., Hold, G. L., Barcenilla, A., Stewart, C. S. & Flint, H. J. Roseburia intestinalis sp. nov., a novel saccharolytic, butyrate-producing bacterium from human faeces. International Journal of Systematic and Evolutionary Microbiology 52, 1615-1620 (2002).
30. Hillman, E. T. et al. Comparative genomics of the genus Roseburia reveals divergent biosynthetic pathways that may influence colonic competition among species. Microbial Genomics 6, 7-24 (2020).
31. Duncan, S. H., Hold, G. L., Harmsen, H. J. M., Stewart, C. S. & Flint, H. J. Growth requirements and fermentation products of Fusobacterium prausnitzii, and a proposal to reclassify it as Faecalibacterium prausnitzii gen. nov., comb. nov. International Journal of Systematic and Evolutionary Microbiology 52, 2141-2146 (2002).
32. Ortiz-Alvarez, L., Xu, H. & Martinez-Tellez, B. Influence of Exercise on the Human Gut Microbiota of Healthy Adults: A Systematic Review. Clin Transl Gastroenterol 11, (2020).
33. Scheiman, J. et al. Meta-omics analysis of elite athletes identifies a performance-enhancing microbe that functions via lactate metabolism. Nature Medicine 25, 1104-1109 (2019).
34. Lin, W.-Y. et al. Probiotic Strains Isolated from an Olympic Woman's Weightlifting Gold Medalist Increase Weight Loss and Exercise Performance in a Mouse Model. Nutrients 14, 1270 (2022).
35. Byeon, H. R. et al. New Strains of Akkermansia muciniphila and Faecalibacterium prausnitzii are Effective for Improving the Muscle Strength of Mice with Immobilization-Induced Muscular Atrophy. Journal of Medicinal Food 25, 565-575 (2022).
36. Ticinesi, A. et al. The gut-muscle axis in older subjects with low muscle mass and performance: a proof of concept study exploring fecal microbiota composition and function with shotgun metagenomics sequencing. International Journal of Molecular Sciences 21, 1-16 (2020).
37. Borgo, F. et al. Microbiota in anorexia nervosa: The triangle between bacterial species, metabolites and psychological tests. PLoS ONE 12, (2017).
38. Huang, C. et al. Distinct Gut Microbiota Composition and Functional Category in Children With Cerebral Palsy and Epilepsy. Frontiers in Pediatrics 7, (2019).
39. Hillman, E. T. et al. Comparative genomics of the genus Roseburia reveals divergent biosynthetic pathways that may influence colonic competition among species. Microbial Genomics 6, 7-24 (2020).
40. Luis Marquezi, M., Augusto Roschel, H., dos Santos Costa, A., Aiko Sawada, L. & Herbert Lancha Jr, A. Effect of Aspartate and Asparagine Supplementation on Fatigue Determinants in Intense Exercise. (2003).
41. Lancha, A. H. et al. Effect of Aspartate, Asparagine, and Carnitine Supplementation in the Diet on Metabolism of Skeletal Muscle During a Moderate Exercise. Physiology & Behavior vol. 57 (1995).
42. Yang, Y. et al. Dietary methionine restriction improves the gut microbiota and reduces intestinal permeability and inflammation in high-fat-fed mice. Food and Function 10, 5952-5968 (2019).
43. Huang, T.-H. et al. Effects of methionine restriction and endurance exercise on bones of ovariectomized rats: a study of histomorphometry, densitometry, and biomechanical properties. J Appl Physiol 119, 517-526 (2015).
44. Swaminathan, A., Fokin, A., Venckunas, T. & Degens, H. Methionine restriction plus overload improves skeletal muscle and metabolic health in old mice on a high fat diet. Scientific Reports 11, (2021).
45. Tomcik K, Ibarra R A, Sadhukhan S, Han Y, Tochtrop G P, Zhang G F. Isotopomer enrichment assay for very short chain fatty acids and its metabolic applications. Anal Biochem. 2011; 410(1):110-117. doi:10.1016/j.ab.2010.11.030.
46. Bodine S C. The role of mTORC1 in the regulation of skeletal muscle mass. Fac Rev. 2022; 11:32. Published 2022 Nov. 11. doi:10.12703/r/11-32.
47. Ben-Sahra I, Hoxhaj G, Ricoult S J H, Asara J M, Manning B D. mTORC1 induces purine synthesis through control of the mitochondrial tetrahydrofolate cycle. Science. 2016; 351(6274):728-733. doi:10.1126/science.aad0489

1. Roseburia inulinivorans for use in improving:
muscular strength;
muscle mass; and/or
pulmonary capacity
in a subject, wherein the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, injury or ageing or combination thereof.

2. Roseburia inulinivorans for use according to claim 1, wherein the Roseburia inulinivorans comprise Roseburia inulinivorans DSM16841.

3. Roseburia inulinivorans for use according to claim 1 or 2, wherein the subject suffers from reduced muscle mass and/or reduced muscle strength.

4. Roseburia inulinivorans for use according to any preceding claim, wherein the muscular condition is selected from the group consisting of:
atony, muscular dystrophy, myasthenia, myotonia, cachexia, sarcopenia, acardiotrophy and frailty, or any combination thereof.

5. Roseburia inulinivorans for use according to any preceding claim, wherein improving muscle strength, muscle mass, and/or pulmonary capacity in the subject:
(i) compensates for wasting resulting from the muscular condition, immobilization, and/or old age; and/or
(ii) is for cosmetic purposes.

6. Roseburia inulinivorans for use according to any preceding claim, wherein the population of Roseburia inulinivorans in the subject is increased by administration of an effective amount of Roseburia inulinivorans to the subject.

7. Roseburia inulinivorans for use according to any preceding claim, wherein the Roseburia inulinivorans in the subject leads to an improvement in one or more of:
handgrip strength;
bench press strength;
leg press strength; and/or
VO2 max;
in the subject.

8. Roseburia inulinivorans for use according to any preceding claim, wherein the Roseburia inulinivorans is administered as part of a composition.

9. Roseburia inulinivorans for use according to claim 8, wherein the composition is a probiotic composition.

10. Roseburia inulinivorans for use according to claim 8, wherein the composition is a postbiotic composition and the Roseburia inulinivorans is non-viable.

11. Roseburia inulinivorans for use according to any one of claims 8 to 10, wherein the composition is a pharmaceutical composition.

12. Roseburia inulinivorans for use according to any one of claims 8 to 11, wherein the composition is comestible.

13. A method of improving or maintaining athletic training, performance or recovery by a subject comprising increasing the population of Roseburia inulinivorans in the subject.

14. A method of improving muscular strength, muscle mass, and/or pulmonary capacity in a subject comprising increasing the population of Roseburia inulinivorans in the subject.

15. The method according to claims 13 or 14, wherein the Roseburia inulinivorans comprise Roseburia inulinivorans DSM16841.

16. The method according to any one of claims 13 to 15, wherein the method is non-therapeutic.

17. The method according to claims 13, 14, or 15, wherein the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, injury or ageing or combination thereof.

18. The method according to any one of claims 13 to 17, wherein the subject suffers from reduced muscle mass and/or reduced muscle strength.

19. The method according to claim 17 or 18, wherein the muscular condition is selected from the group consisting of:
atony, muscular dystrophy, myasthenia, myotonia, cachexia, sarcopenia, acardiotrophy and frailty, or any combination thereof.

20. The method according to any one of claims 13 to 19, wherein the method improves muscular strength, muscle mass, and/or pulmonary capacity in the subject for cosmetic purposes.

21. The method according to any one of claims 13, 14, 15 and 17 to 20, wherein the method improves muscular strength, muscle mass, and/or pulmonary capacity in the subject to compensate for muscle wasting resulting from the muscular condition, immobilization, and/or old age.

22. The method according to any one of claims 13 to 21, wherein the method improves one or more of:
handgrip strength;
bench press strength;
leg press strength; and/or
VO2 max;
in the subject.

23. The method according to any one of claims 13 to 22, wherein the method comprises administering to the subject an effective amount of Roseburia inulinivorans.

24. The method according to any one of claims 13 to 23, wherein the method comprises administering to the subject an effective amount of Roseburia inulinivorans as part of a composition.

25. The method according to claim 24, wherein the composition is a pharmaceutical composition and/or a probiotic composition or postbiotic composition comprising inactivated Roseburia inulinivorans.

26. The method according to any one of claims 13 to 25, wherein the method comprises administering to the subject an effective amount of a composition configured to increase the population of endogenous Roseburia inulinivorans in the subject.

27. The method according to claim 26, wherein the composition configured to increase the population of endogenous Roseburia inulinivorans in the subject comprises a prebiotic.

28. The method according to any one of claims 24 or 27, wherein the composition is comestible.

29. Use of Roseburia inulinivorans for improving in a subject:
muscular strength;
muscle mass; and/or
pulmonary capacity.

30. Use of Roseburia inulinivorans for improving or maintaining athletic training, performance or recovery by a subject.

31. Use of Roseburia inulinivorans for the manufacture of a medicament for:
improving in a subject:
muscular strength;
muscle mass; and/or
pulmonary capacity; or
improving or maintaining athletic training, performance or recovery by a subject.

32. The use according to any one of claims 29 to 31, wherein the Roseburia inulinivorans comprise Roseburia inulinivorans DSM16841.

33. A prebiotic composition for increasing the population of endogenous Roseburia inulinivorans in a subject for use in improving:
muscular strength;
muscle mass; and/or
pulmonary capacity
in the subject, wherein the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, injury or ageing or combination thereof.

34. The prebiotic composition for use according to claim 33, wherein the Roseburia inulinivorans comprise Roseburia inulinivorans DSM16841.

35. The prebiotic composition for use according to claim 33 or 34, wherein the subject suffers from reduced muscle mass and/or reduced muscle strength.

36. The prebiotic composition for use according to claims 33 or 35, wherein the muscular condition is selected from the group consisting of:
atony, muscular dystrophy, myasthenia, myotonia, cachexia, sarcopenia and frailty, or any combination thereof.

37. The prebiotic composition for use according to any one of claims 33 to 36 wherein improving muscle strength, muscle mass, and/or pulmonary capacity in the subject:
(i) compensates for wasting resulting from the muscular condition, immobilization, and/or old age; and/or
(ii) is for cosmetic purposes.

38. The prebiotic composition for use according to any one of claims 33 to 37, wherein the increased population of Roseburia inulinivorans in the subject leads to an improvement in one or more of:
handgrip strength;
bench press strength;
leg press strength; and/or
VO2 max;
in the subject.

39. A postbiotic composition comprising:
a culture extract of a culture of Roseburia inulinivorans.

40. The postbiotic composition of claim 39, wherein the culture extract comprises non-viable Roseburia inulinivorans or a part thereof.

41. The postbiotic composition of claims 39 or 40, wherein the culture extract comprises compounds extracted and/or secreted from Roseburia inulinivorans.

42. The postbiotic composition of any one of claims 39 to 41, wherein the Roseburia inulinivorans comprises Roseburia inulinivorans DSM16841.

43. A method of improving or maintaining athletic training, performance or recovery by a subject comprising administering a postbiotic composition according to any one of claims 39 to 42.

44. A method of improving muscular strength, muscle mass, and/or pulmonary capacity in a subject comprising administering a postbiotic composition according to any one of claims 39 to 42.

45. A postbiotic composition according to any one of claims 39 to 44, for use in improving:
muscular strength;
muscle mass; and/or
pulmonary capacity
in a subject, wherein the subject suffers from a muscular condition selected from the group consisting of: atony, a muscular dystrophy, myasthenia, myotonia, cachexia, sarcopenia, acardiotrophy, frailty and/or reduced muscle mass and/or reduced muscle strength caused by a sequelae of immobilization, chronic disease, cancer, injury or ageing or combination thereof.

46. The postbiotic composition for use according to claim 45 or the method according to any one of claims 43 or 44, wherein the subject suffers from a muscle-related disorder, reduced muscle mass and/or reduced muscle strength.

47. The postbiotic composition for use according to claims 45 or 46 or the method according to any one of claims 43, 44 or 46, wherein the muscular condition is selected from the group consisting of:
atony, muscular dystrophy, myasthenia, myotonia, cachexia, sarcopenia and frailty, or any combination thereof.

48. The postbiotic composition for use according to any one of claims 45 to 47 or the method according any one of claims 43, 44, 46 or 47, wherein improving muscle strength, muscle mass, and/or pulmonary capacity in the subject:
(i) compensates for wasting resulting from the muscular condition, immobilization, and/or old age; and/or
(ii) is for cosmetic purposes.

49. The postbiotic composition for use according to any one of claims 45 to 48 or the method according any one of claims 43, 44, or 46 to 48, wherein the postbiotic leads to an improvement in one or more of:
handgrip strength;
bench press strength;
leg press strength; and/or
VO2 max;
in the subject.

50. The method of any of claims 26 to 28 wherein the composition comprises a prebiotic composition or the prebiotic composition for use according to any of claims 33 to 38, wherein the prebiotic composition comprises one or more of:
inulin;
melibiose;
glucose;
sucrose;
starch;
acetate;
lactate;
butyrate;
propionate;
fructo-oligosaccharides;
fucose; and/or
methionine.


SEQUENCE LISTING (2 sequences)

SEQ ID NO: 1 - DNA, 17 bp, synthetic construct
  source 1..17: mol_type = other DNA
cctacgggnggcwgcag

SEQ ID NO: 2 - DNA, 21 bp, synthetic construct
  source 1..21: mol_type = other DNA
gactachvgggtatctaatcc